(19) United States
(12) Patent Application Publication
(10) Pub. No.: US 2016/0361462 A1
(43) Pub. Date: Dec. 15, 2016

(54) BIOACTIVE MODIFICATION OF POLY(VINYL ALCOHOL) WITH SURFACE TOPOGRAPHY AND BIOCHEMICAL CUES FOR VASCULAR GRAFT

(71) Applicants: NATIONAL UNIVERSITY OF SINGAPORE, Singapore (SG); INSTITUT NATIONAL DE LA SANTE ET DE LA RECHERCHE MEDICALE (INSERM), Paris Cedex 13 (FR)

(72) Inventors: King Fai Evelyn YIM, Singapore (SG); Catherine LE VISAGE, Paris (FR); Marie Francene Arnobit CUTIONGCO, Singapore (SG); Ming Hao TAN, Singapore (SG); Seok Hong GOH, Singapore (SG)

(73) Assignees: NATIONAL UNIVERSITY OF SINGAPORE, Singapore (SG); INSTITUT NATIONAL DE LA SANTE ET DE LA RECHERCHE MEDICALE (INSERM), Paris Cedex 13 (FR)

(21) Appl. No.: 15/097,749

(22) Filed: Apr. 13, 2016

Related U.S. Application Data
(60) Provisional application No. 62/147,026, filed on Apr. 14, 2015.

(51) Int. Cl. A61L 27/16; A61L 27/54; C08J 7/12

(52) U.S. Cl. CPC A61L 27/16; C08J 7/12; A61L 27/54; C08J 2329/04; A61L 2300/414; A61L 27/50

(57) ABSTRACT

Modifications of the biomaterial poly(vinyl alcohol) with surface topographical cues, attachment factors for its improved performance, and/or sustained release of vascular endothelial biochemical cue for application as a vascular graft scaffold is described. Furthermore, novel fabrication methods to pattern the poly(vinyl alcohol) hydrogel in planar film or tubular form with the topographies in the lumen are disclosed.

### BACKGROUND OF THE INVENTION

Peripheral arterial disease (PAD) is a leading cause of morbidity worldwide, afflicting 27 million people in Europe and the United States. Though not fatal, PAD severely reduces functional capacity and overall quality of life in all patients. When faced with the need for surgical revascularization of the afflicted limb, autologous blood vessels are required [Brevetti G, et al., Atherosclerosis 197, 1-11 (2008); Curi M et al., Annals of Vascular Surgery 16, 95-101(2002); Michaels J, British Journal of Surgery 76, 7-14(1989)]. Most PAD patients, however, present with other comorbidities that increase the risk of an additional surgical procedure. Synthetic small diameter vascular grafts (SDVG), with internal diameter of less than 6 mm, are then used as an alternative. SDVGs available today are limited by thrombogenicity, induction of intimal hyperplasia and overall lack of long-term patency [Venkatraman S, et al., Progress in Polymer Science 33, 853-874 (2008)].

The occlusion of arteries often leads to ischemia, thus requiring vascular replacement. Surgical bypass and replacement of occluded peripheral arteries (internal diameter of less than 6 mm) is a major choice of surgical treatment. Ideally, autologous vessel grafts are used for this purpose because it prevents biocompatibility problems and achieve high graft patency rates [Salacinski H. J., et al., Journal of Biomaterial Applications 15, 241-78 (2001)]. Thus, the autologous saphenous vein and mammary artery remain the gold standard for use as vascular grafts in bypass surgery [Lamm P., et al., Circulation 18, 1108-14 (2001); Beard J. D., Journal of Vascular Surgery 48, 11S-16S (2008)]. Unfortunately, such graft sources are scarce as autologous veins are not available or inadequate in a majority of patients due to the typical presence of coexisting diseases [Salacinski H. J., et al., Journal of Biomaterial Applications 15, 241-78 (2001)]. In addition, the extra procedure of harvesting healthy vessel increases the morbidity and mortality of such a procedure. The alternative is to use synthetic materials, which are limited in application due to its thrombogenicity and low long-term patency. This problem is much more severe in small diameter synthetic grafts, with internal diameter of 6 mm or less.

Tissue-engineered grafts were thus proposed to counter the abovementioned problems and yet maintain the biocompatibility and patency standards of the autologous vessel. However, naturally-derived biomaterials still pose problems of having thrombogenic properties. On the other hand, synthetic grafts are a good alternative as they are readily available, easily manufactured with the desired mechanical properties and relatively cheap. Since the introduction of synthetic materials for vascular grafts in the 1950s, a variety of materials have been applied and compared as vascular graft candidates, with polytetrafluoroethylene (PTFE) and Dacron emerging the most widely-used materials today. PTFE is a relatively stiff and inert fluorocarbon polymer, prepared and used as expanded PTFE by extrusion and sintering, while Dacron is a fibrous polyester that can be woven or knitted into grafts. Both materials are bioinert and do not interact with tissue, thus are used with excellent results in lower limb bypass grafts (7-9 mm). However, PTFE and Dacron grafts have poor patency and rapidly occlude when used in small-caliber arteries.

Various studies have modified materials with biochemical cues to make them more amenable for tissue engineering applications. It is also common to modify surfaces to directly present attachment factors that will anchor cells on to a scaffold. For instance, various surfaces have been modified with fibronectin, or its active tripeptide moiety, RGD. Additionally, use of a chemotactic and mitogenic factor can provide the proper signals for cellular infiltration and attachment to the scaffold. For example, vascular endothelial growth factor (VEGF), one of the most potent biochemical cues for endothelial cell migration and proliferation and angiogenesis, is commonly used for scaffold modification.

Poly(vinyl alcohol) (PVA) is a biocompatible and non-thrombogenic water soluble polymer. The hydrophilicity of PVA gives rise to its excellent blood compatibility and its refractory effect on endothelial cell adhesion. A preliminary study by Chaouat et al (2008) [Advanced Functional Materials 18, 2855-2861 (2008)] disclosed a cross-linking method without the use of organic solvent or extreme temperatures, demonstrating good mechanical properties and short-term patency of PVA in the rat aorta, though without endothelialisation.

### SUMMARY OF THE INVENTION

The present invention is directed to methods to control and customize the properties of a SDVG by including nano- and micro-patterns on PVA film using aqueous PVA solution that is crosslinked without the use of organic solvent. Furthermore, a small diameter PVA scaffold with topography was fabricated to improve in situ endothelialisation and patency. The functionality of PVA was further enhanced by surface modification with attachment factor and sustained release of VEGF, the VEGF-mimic QK, an alternative chemotactic factor for endothelial cells, and AcSDKP (N-acetyl Ser-Asp-Lys-Pro).

Patterned PVA films were fabricated to determine the patternability of the PVA polymer. PVA was crosslinked with sodium trimetaphosphate (STMP) and NaOH in aqueous solution. A casting method was used for the fabricating patterned planar films. Tubular scaffolds, either without pattern or patterned with anisotropic patterns (gratings) or isotropic patterns (lens and pillars), were fabricated through a novel dip-casting method. The micro gratings are, for example, 2 μm×2 μm×2 μm, or 10 μm×10 μm×10 μm ridge×gap×height. The luminal surface of the PVA tubular scaffolds was verified to contain gratings of good integrity, demonstrating the success of the novel patterning process. Cell viability and function was enhanced on patterned PVA films.

PVA planar films were also modified with attachment factors to improve its cell adhesion capacity. A novel method for crosslinking attachment factors to PVA was performed, in which biochemical factors heparin, fibronectin, RGDS tetrapeptide or cyclic RGD peptide (cRGD) was mixed into the activated PVA solution. Cell attachment and function improved on modified PVA films.

Bioactive growth factor and peptides, VEGF and PEGylated QK (QK-PEG) and AcSDKP, were encapsulated through the use of interfacial polyelectrolyte complexation (IPC) fibers, which have been shown to act as a biologics reservoir that controls release spatially and temporally. We have shown the sustained release of both VEGF and QK-PEG from PVA-IPC fiber composite tubular scaffold. Furthermore, we demonstrated the retained biological activity of the released growth factors through its stimulation of endothelial cell proliferation.

In a first aspect of the invention there is provided A bioactive scaffold, comprising:

poly(vinyl alcohol) scaffold and at least one modification at a surface of the scaffold, the at least one modification comprising a surface topographical cue and/or an attachment factor.

In a preferred embodiment the scaffold is a small diameter vascular graft.

In another preferred embodiment the surface topographical cue is an anisotropic pattern or an isotropic pattern.

In another preferred embodiment the surface topographical cue is an indentation in the surface or a protrusion from the surface of the scaffold.

In another preferred embodiment the surface topographical cue has a depth differential of one nanometer to 50 micrometers relative to the surface of the scaffold.

In another preferred embodiment the attachment factor is selected from the group comprising a peptide, a polysaccharide, a protein, a nucleic acid, an oligonucleotide, a nanoparticle, an organic small molecule, or an inorganic compound.

In another preferred embodiment the attachment factor is selected from the group comprising heparin, fibronectin, Arg-Gly-Asp-Ser (RGDS) or cyclo(Cys-Arg-Arg-Gly-Asp-Trp-Leu-Cys) (cRGD).

Another preferred embodiment of the scaffold of the invention further comprises an interfacial polyelectrolyte complexation (IPC) fiber and a biologic.

As used herein the term ‘polyelectrolyte complexation (PEC) fiber’ is also known as, and used interchangeably with, the term ‘interfacial polyelectrolyte (polyion) complexation (IPC) fiber’. Interfacial polyelectrolyte (polyion) complexation (IPC) is a process whereby fibers and capsules are formed through interactions at the interface of oppositely charged polymers [for example, see Liao, I. C., et al., J Control Release 104(2), 347-58 (2005)].

In another aspect of the invention there is provided a bioactive scaffold comprising poly(vinyl alcohol), a IPC fiber and a biologic.

In a preferred embodiment the biologic is encapsulated in a matrix comprising the IPC fiber, such that, in use, the matrix sustainably and controllably releases the biologic.

In another preferred embodiment a matrix comprising the IPC fiber is enclosed between one or more layers of poly(vinyl alcohol).

In another preferred embodiment the biologic is selected from the group comprising a growth factor, an enzyme, a peptide, a cell, an antibody, an antioxidant, an angiogenic molecule, an antiangiogenic molecule, an immune-modulatory molecule, a pro-inflammatory molecule, an anti-inflammatory molecule, a nucleic acid, an oligonucleotide, an adhesion molecule, or a pharmaceutical composition.

In another preferred embodiment the biologic is vascular endothelial growth factor (VEGF).

In another aspect of the invention there is provided a method for controlling the release of a biologic from a bioactive scaffold, comprising:

providing a bioactive scaffold comprising poly(vinyl alcohol), a IPC fiber and a biologic, wherein the composition and fabrication of the scaffold is selected to control the release of the biologic; and

exposing the bioactive scaffold to conditions in which release of the biologic is induced.

In another aspect of the invention there is provided a method of fabricating a bioactive scaffold, comprising:

(a) providing a tubular mold, optionally comprising a topographically patterned outer surface;

(b) contacting the tubular mold of step (a) with an aqueous solution comprising poly(vinyl alcohol) to form a tube coated with poly(vinyl alcohol);

(c) drying the coated tube of step (b) to evaporate residual water;

(d) repeating steps (b) and (c) as necessary to achieve a desired thickness of the bioactive scaffold.

In a preferred embodiment the method of fabricating a bioactive scaffold additionally comprising steps (e) and (f), wherein steps (e) and (f) are performed after any iteration of step (c), and further wherein steps (e) and (f) are:

(e) wrapping a fibrous matrix comprising IPC fibers onto the outer surface of the coated tube to form a fiber-wrapped coated tube; and

(f) contacting the fiber-wrapped coated tube of step (e) with an aqueous solution comprising poly(vinyl alcohol) to form a layered tube with an outer coating of poly(vinyl alcohol).

In a preferred embodiment the method of fabricating a bioactive scaffold the fibrous matrix further comprises a biologic.

In a preferred embodiment the biologic is vascular endothelial growth factor (VEGF).

In another aspect of the invention there is provided a bioactive scaffold produced according to the method of any aspect of the invention.

In another aspect of the invention there is provided a method of treatment comprising administering to a subject in need of such treatment a bioactive scaffold as defined herein.

In a preferred embodiment, the treatment is vascular repair.

In another aspect of the invention there is provided a kit for vascular repair comprising a bioactive scaffold as defined herein.

### BRIEF DESCRIPTION OF THE DRAWINGS

FIGS. 1A, 1B and 10 depict implantation of PVA in a rabbit model of peripheral arterial disease (PAD). (FIG. 1A) Representative PVA small diameter vascular graft with internal diameter 1 mm. (FIG. 1B) PVA vascular graft anastomosed to rabbit femoral artery. (FIG. 10) Digital subtraction angiography at 15 days post-implantation shows patency of PVA vascular graft. Arrow head shows location of the PVA vascular graft.

FIGS. 2A, 2B, 2C and 2D depict endothelialization of PVA small diameter vascular graft. (FIG. 2A, FIG. 2C) No positive stain for CD31 was found on nonimplanted PVA vascular grafts. (FIG. 2B, FIG. 2D) Endothelial cells, as detected by immunohistochemistry using CD31 marker, was found on the lumen of PVA small diameter vascular grafts after 15 days.

FIGS. 3A, 3B, 3C and 3D show the casting method to create planar PVA film with topography. (FIG. 3A) A piece of tissue culture polystyrene (TOPS) heat embossed with the isotropic gratings was used as a template. (FIG. 3B-3C) Subsequently, the activated PVA solution was poured on top of the pattern and centrifuged for 30 mins at 1000 rpm. PVA was then allowed to degas for 3 hours, and subsequently centrifuged again for 30 mins at 1000 rpm. (FIG. 3D) PVA was allowed to be crosslinked slowly at 18° C. for 5 days. After curing, the PVA is demolded from the TOPS pattern by using distilled deionized water.

FIGS. 4A, 4B, 4C, 4D, 4E, 4F, 4G, 4H, 4I and 4J show scanning electron microscopy (SEM) images of planar PVA films with different topographies. The demolded PVA films were air-dried overnight before imaging. Inserts show high magnification images. PVA was patterned with (FIG. 4A) 250 nm gratings, (FIG. 4B) 2 μm gratings, (FIG. 4C) 10 μm gratings, (FIG. 4D) 2 μm Pillars, (FIG. 4E) 10 μm Concave microlens in square array, (FIG. 4F) Concave microlens with diameter of 1.8 μm, pitch of 2 μm and sag of 0.7 μm, (FIG. 4G) Convex microlens with diameter of 1.8 μm, pitch of 2 μm and sag of 0.7 μm, (FIG. 4H) 10 μm Pillars, (FIG. 4I) 2 μm Concave microlens in square array, (FIG. 4J) Unpatterned.

FIGS. 5A and 5B show surface analysis of planar PVA obtained via casting method. (FIG. 5A) X-ray photoelectron spectroscopy (XPS) analysis of unpatterned PVA shows characteristic carboxyl, carbonyl, hydroxyl and methyl groups. (FIG. 5B) Water contact angle analysis of PVA films with topography obtained via casting. * denotes significant difference at p<0.05.

FIGS. 6A and 6B demonstrate improvement of endothelial cell viability on planar PVA with surface topography. (FIG. 6A) Cell viability is given as ratio fluorescent signal from live (calcein-AM positive cells) over dead (Ethidium homodimer-1 positive cells) cells. (FIG. 6B) Proliferation rate is given as percentage of cells with EdU uptake. Endothelial cells had better viability when cultured on patterned PVA films after 24 hours. Error bars represent standard deviation.

FIGS. 7A, 7B, and 7C show endothelial cell adhesion on patterned PVA obtained through casting method. (FIG. 7A) Representative images showing morphology of human endothelial cells on various patterned planar PVA. PVA with the following patterns were tested: (i) unpatterned, (ii) 10 μm gratings, (iii) 2 μm gratings, (iv) 10 μm pillars, (v) 2 μm pillars, (vi) 10 μm convex lens, (vii) 2 μm convex lens, (viii) 1.8 μm convex lens, (ix) 1.8 μm concave lens. (FIG. 7B) Total number of cells adhered to various patterned planar PVA. (FIG. 7C) Area of cells on PVA with selected topographies.

FIGS. 8A, 8B, 8C, 8D, 8E, and 8F show retention of endothelial cell phenotype grown on patterned PVA films. PVA films with 2 μm gratings (FIG. 8A), 10 μm gratings (FIG. 8B), Convex microlens (FIG. 8C), Concave microlens (FIG. 8D), and Unpatterned (FIG. 8E) were used. Glass coverslip was used as a control (FIG. 8F). Endothelial cells were grown for 24 hours on PVA films and subsequently fixed for immunofluorescence detection of endothelial phenotype marker CD31 (arrows). Grating axis is denoted by the white arrow at the bottom right of each image. Nuclei were detected using DAPI. Scale bar=50 μm.

FIGS. 9A, 9B, 9C, 9D, 9E, and 9F show retention of endothelial cell function on patterned PVA films with 2 μm gratings (FIG. 9A), 10 μm gratings (FIG. 9B), Convex microlens (FIG. 9C), Concave microlens (FIG. 9D), and Unpatterned (FIG. 9E) were used. Glass coverslip was used as a control (FIG. 9F). Endothelial cells were grown for 24 hours on PVA films and subsequently fixed for immunofluorescence detection of endothelial phenotype marker vWF (arrow). Grating axis is denoted by the white arrow at the bottom right of each image. Nuclei were detected using DAPI. Scale bar=50 μm.

FIGS. 10A, 10B, 10C, 10D, 10E and 10F illustrate retention of in vitro angiogenic capacity of cells grown on patterned planar PVA film. PVA films with 2 μm gratings (FIG. 10A), 10 μm gratings (FIG. 10B), Convex microlens (FIG. 10C), Concave microlens (FIG. 10D), and Unpatterned (FIG. 10E) were used. Glass coverslip was used as a control (FIG. 10F). After endothelial cells were grown for 24 hours on the various PVA films, they were harvested and seeded onto Matrigel to assess tubular formation and thus angiogenic capacity. Cells were grown on Matrigel for 8 hours. Scale bar=200 μm.

FIGS. 11A, 11B, 11C, 11D and 11E illustrate the dip-casting method for creating tubular PVA scaffold with topography on the luminal surface. (FIG. 11A) Patterned PLLA or PDMS film was wrapped around a tubular mold with the patterned surface facing outward. (FIG. 11B) The mold was plasma treated and dipped into activated PVA solution. (FIG. 11C) Afterwards, the mold and activated PVA solution was sonicated for 1 hour to improve quality of patterning. (FIG. 11D) Thereafter, the mold was dipped several times into a new activated PVA solution to obtain the desired thickness and mechanical properties. (FIG. 11E) The molds were slowly dried for 3 days at 18° C. then demolded using distilled deionized water.

FIGS. 12A, 12B, 12C, 12D, 12E, 12F, 12G and 12H demonstrate patterning of tubular PVA scaffolds with luminal surface topography. (FIG. 12A-E) PVA vascular graft (2-2.25 mm diameter) with topography incorporated in the luminal surface. PVA vascular grafts were patterned with 2 μm gratings (FIG. 12A), 10 μm gratings (FIG. 12B), Convex microlens (FIG. 12C), Concave microlens (FIG. 12D), and without patterns (FIG. 12E). Inset shows high magnification SEM. (FIG. 12F) Prototype of PVA vascular graft with 7 cm length and 4 mm diameter. SEM shows that long PVA grafts had 2 μm gratings (FIG. 12G) and convex microlens (FIG. 12H) topography on the luminal surface.

FIGS. 13A, 13B, 13C, 13D, 13E, 13F, 13G and 13H show in vivo implantation of patterned PVA vascular grafts in rat abdominal aorta model. (FIG. 13A) Overview of PVA vascular graft implanted in rat abdominal aorta. (FIG. 13B) Hematoxylin and eosin (H&E) staining of PVA vascular graft with 2 μm gratings. (FIG. 13C) Black arrows denote retention of 2 μm gratings on the PVA vascular graft 20 days post-implantation. (FIG. 13D) Immunofluorescence staining shows RECAP cells (red) on the luminal surface of the PVA vascular graft with 2 μm gratings. Nuclei are stained with DAPI (blue). White arrow shows the endothelial cell layer. A neointimal layer was also observed. (FIG. 13E) H&E staining of cRGD-PVA graft with microlens. (FIG. 13F) Immunofluorescence staining shows RECAP cells (red) on the luminal surface of the graft. Nuclei are stained with DAPI (blue). White arrow shows the endothelial layer. H&E staining of unpatterned PVA (FIG. 13G) and unpatterned cRGD-PVA (FIG. 13H) vascular grafts.

FIGS. 14A, 14B, 14C and 14D show modification of planar PVA with biochemical cues Crgd (FIG. 14A), RGDS (FIG. 14B), protein Fibronectin (FIG. 14C) and polysaccharide heparin (FIG. 14D). SEM images show roughness of PVA films modified with biochemical cues. Inset shows higher magnification image of heparin-PVA films.

FIGS. 15(A)-(B) demonstrate improvement of endothelial cell viability on planar PVA modified with biochemical cues. (FIG. 15A) Cell viability is given as ratio of fluorescent signal from live (calcein-AM positive cells) over dead (Ethidium homodimer-1 positive cells) cells. (FIG. 15B) Proliferation rate is given as percentage of cells with EdU uptake. Endothelial cells had better viability when cultured on modified PVA films. Heparin-PVA did not have a positive effect on endothelial cell viability and proliferation.

FIGS. 16A, 16B, 16C and 16D demonstrate retention of endothelial cell phenotype grown on PVA films modified with Crgd (FIG. 16A), RGDS (FIG. 16B), Fibronectin (FIG. 16C) and Heparin (FIG. 16D). Endothelial cells were grown for 24 hours on PVA films and subsequently fixed for immunofluorescent detection of CD31 (green; light). Nuclei were detected by DAPI (dark). Grating axis is denoted by the white arrow at the bottom right of each image. Scale bar=50 μm.

FIGS. 17A, 17B, 17C and 17D demonstrate retention of endothelial cell function on PVA films modified with cRGD (FIG. 17A), RGDS (FIG. 17B), Fibronectin (FIG. 17C), and Heparin (FIG. 17D). Endothelial cells were grown for 24 hours on PVA films and subsequently fixed for immunofluorescence detection of vWF (red). Nuclei were detected by DAPI. More endothelial cells retain expression of CD31 and vWF after seeding on modified PVA films. vWF punctuate staining is highest on heparin and RGDS-modified films. Grating axis is denoted by the white arrow at the bottom right of each image. Scale bar=50 μm.

FIGS. 18A, 18B, 18C and 18D show retention of in vitro angiogenic capacity of cells grown on planar PVA film modified with cRGD (FIG. 18A), RGDS (FIG. 18B), Fibronectin (FIG. 18C), and Heparin (FIG. 18D). After endothelial cells were grown for 24 hours on the various PVA films, they were harvested and seeded onto Matrigel to assess tubular formation and thus angiogenic capacity. Cells were grown on Matrigel for 8 hours. Tubular formation was enhanced in endothelial cells that were grown on RGDS and fibronectin modified PVA films. Scale bar=200 μm.

FIGS. 19A, 19B, 19C and 19D illustrate patterning of modified PVA films. Casting method was used to pattern cRGD-modified PVA films with 2 μm gratings (FIG. 19A), 10 μm gratings (FIG. 19B), Convex microlens (FIG. 19C) and Concave microlens (FIG. 19D), thereby introducing both topographical and biochemical cues in one planar PVA scaffolds.

FIGS. 20A and 20B show improved endothelial cell viability on cRGD-PVA with topography. (FIG. 20A) cRGD-PVA with topography supported cell adhesion and normal endothelial morphology (i) 2 μm gratings, (ii) 10 μm pillars, (iii) 10 μm lens, (iv) without pattern, (v) 2 μm pillars, (vi) concave microlens, (vii) convex microlens were tested. (viii) PVA films without pattern and without cRGD were used as a control. (FIG. 20B) Patterned cRGD-PVA increased cell adhesion.

FIGS. 21A and B show that topographical cues and biochemical cues improve blood compatibility by decreasing platelet activation and adhesion. (FIG. 21A) SEM showed platelets had less activated morphology on PVA with topography compared to platelets on unpatterned PVA or glass. Incorporation of cRGD into PVA films further improved platelet morphology. (FIG. 21B) Microparticle release was measured to assess platelet activation. cRGD-PVA with 2 μm gratings showed largest decrease while cRGD-PVA with convex microlens showed largest increase in number of adhered platelets compared with their respective patterned PVA controls.

FIG. 22A shows improvement of endothelial cell attachment on PVA substrates treated with plasma. Endothelial cell adhesion on unpatterned PVA substrates extensively after plasma treatment.

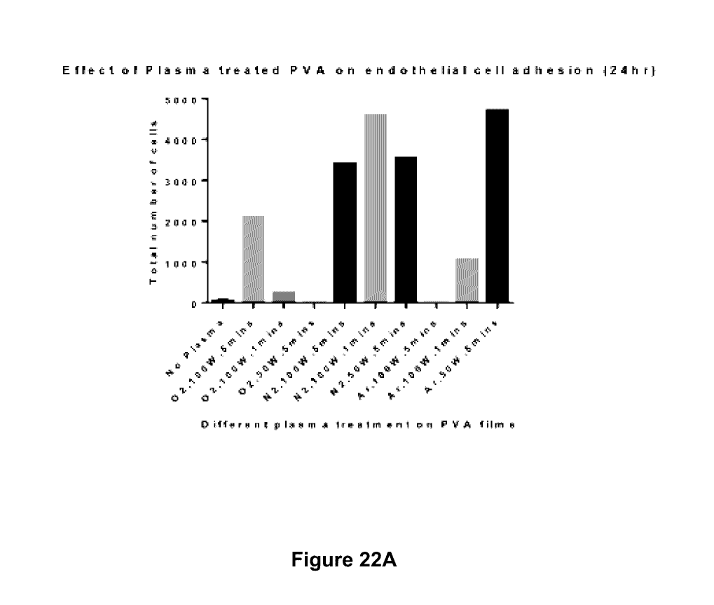

FIG. 22B shows representative images of endothelial cell attachment on PVA substrates treated with (i) Oxygen (O₂) gas, (ii) Nitrogen (N₂) gas, (iii) Argon (Ar) gas. All substrates shown were treated at 100 W for 5 minutes. (iv) PVA substrate without plasma treatment were included as a control.

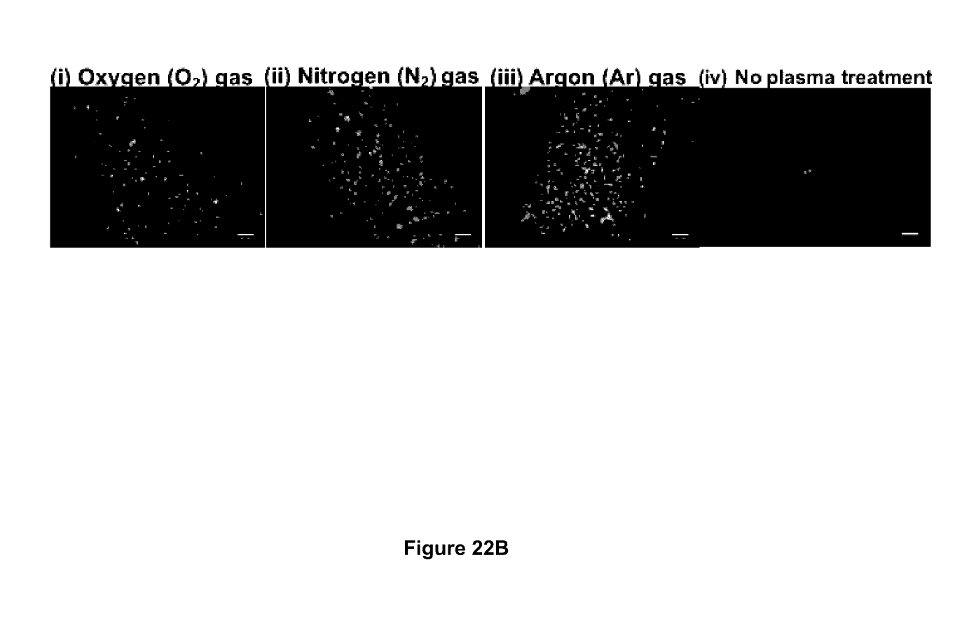

FIGS. 23A, 23B and 23C demonstrates N2 gas plasma modification of planar PVA (i) without topography, (ii) 2 μm gratings, (iii) 2 μm pillars, (iv) 1.8 μm convex lens, (v) 1.8 μm concave lens topography. (FIG. 23A) SEM of patterned PVA after N2 gas plasma modification at 50 W for 1 minute. (FIG. 23B) XPS shows changes in the surface composition of unpatterned planar PVA after N2 gas plasma modification. (FIG. 23C) Water contact angle measurements of patterned PVA after N2 gas plasma modification.

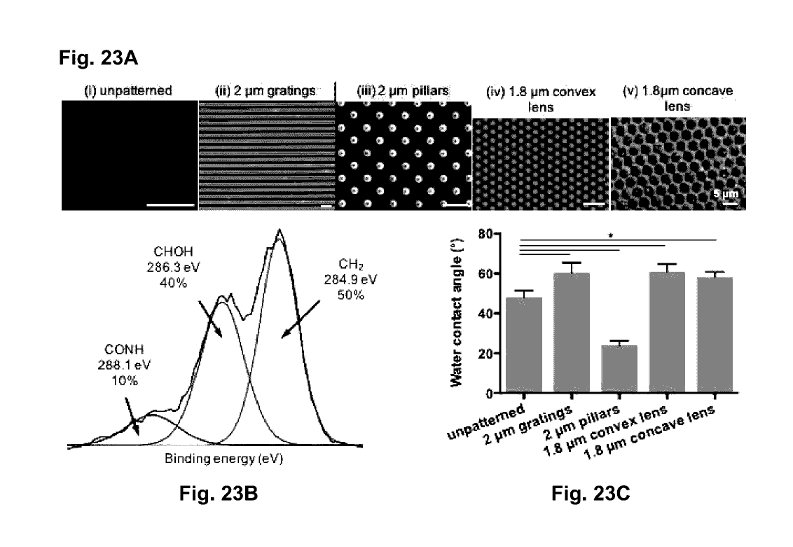

FIGS. 24A and 24B shows significant enhancement of endothelial cell attachment on patterned PVA substrates after plasma treatment. All PVA substrates were plasma treated with nitrogen (N2) gas for 1 mins at 50 W. (FIG. 24A) Representative images showing morphology of human endothelial cells on N2 gas plasma modified PVA. Green denotes actin cytoskeleton and blue denotes nuclei. Plasma treated PVA films (i) without topography, (ii) 2 μm gratings, (iii) 2 μm pillars, (iv) 1.8 μm convex lens, (v) 1.8 μm concave lens were tested. (vi) Glass coverslip were used as a control. (FIG. 24B) Total cell adhesion on N2 gas plasma after 24 hours. *denotes statistical significance against unpatterned using two-way ANOVA.

FIG. 25(A) shows monolayer formation on N2 gas plasma modified PVA with topography. Representative images of human endothelial monolayer on patterned PVA with N2 gas plasma modification. Plasma treated PVA films (i, vii) without topography, (ii,viii) 2 μm gratings, (iii, ix) 2 μm pillars, (iv, x) 1.8 μm convex lens, (v, xi) 1.8 μm concave lens were tested. (vi, xii) Glass coverslip were used as a control. Red denotes the actin cytoskeleton, green denotes the cell-cell junction marker Vascular Endothelial cadherin (VE-cadherin) and blue denotes the nucleus.

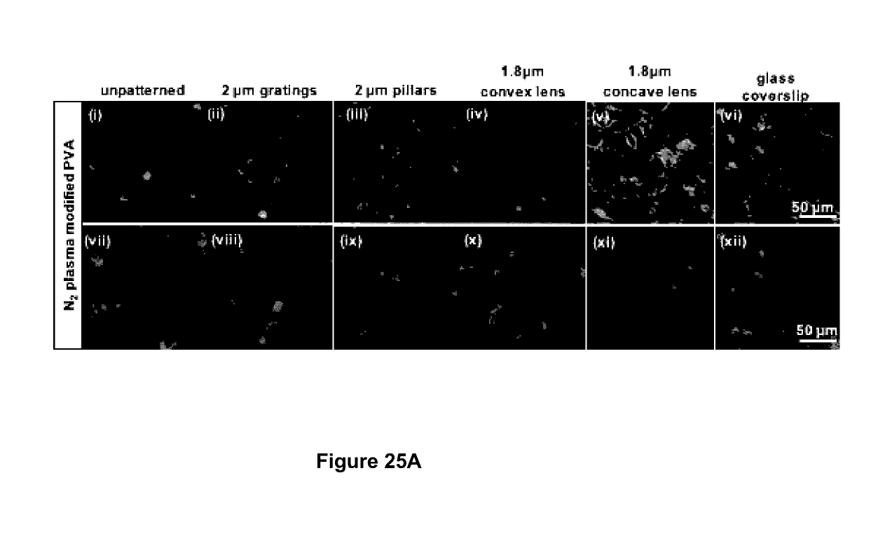

FIG. 25(B) Semi-quantitative analysis of VE-cadherin expression. *denotes statistically significant difference with two-way ANOVA.

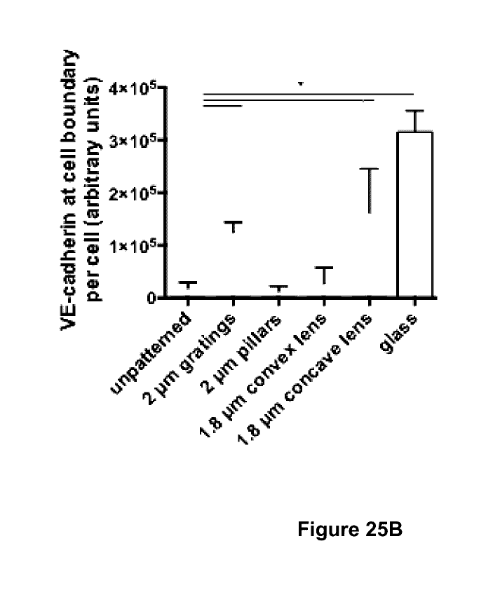

FIG. 26 shows advantages of PVA vascular graft with controlled release. Schematic diagram to show therapeutic effect of vascular graft with controlled release in peripheral arteries. (i) Enables local release of heparin or anti-coagulant to prevent further thrombosis. Local administration of drug will decrease in systemic dose and reduce potential side-effects while ensuring therapeutic action locally. (ii) Release of growth factor to treat microvascular occlusion at distal arterial sites.

FIG. 27(A)-(C) shows a method for the incorporation of interfacial polyelectrolyte complexation (IPC) fibers to create a PVA-IPC fiber composite scaffold. (A) A mold with 4 layers of PVA was fabricated and allowed to dry (inner layer). IPC fibers, encapsulating the biologics of interest, was spun around the inner layer and allowed to dry. (B) Afterwards, 2 more PVA layers are added on the outside (outer layer). (C) The outer layer of PVA was allowed to dry before demolding using distilled deionized water.

FIGS. 28A and 28B depict various modalities of IPC fibers incorporated on IPC-PVA composite scaffolds. (FIG. 28A) Different orientations of IPC fibers that can be incorporated in between PVA layers (i) Longitudinal, (ii) Angular, (iii) Circumferential and (iv) Bi-directional angular IPC fiber orientations. (FIG. 28B) Example of specific mechanism for incorporating Angular and Bi-directional Angular IPC fibers. (i) Using a platform to actuate the PVA tubular scaffold, the IPC fibers were oriented at a specific angle with respect to the PVA scaffold. (ii) The speed of the actuator and the rotation of the PVA tubular scaffold determined the angle of the angular IPC fibers. (iii) By actuating the PVA scaffold in the opposite direction with the same rate, bi-directional angular IPC fibers were obtained.

FIGS. 29A, 29B and 29C demonstrate incorporation of IPC fibers in PVA tubular scaffolds for controlled release of biologics. (FIG. 29A) PVA tubular scaffold made of 6 layers of PVA and without any IPC fibers. (FIG. 29B) PVA scaffold with circumferential IPC fibers in between 4 inner layers and 2 outer layers of PVA. Red arrows demarcate the start and end of the layer containing the IPC fibers. (FIG. 29C) PVA scaffold with biangular IPC fibers.

FIGS. 30A and 30B show controlled release of lysozyme (14 kDa) from IPC fibers incorporated into PVA planar film or PVA tubular scaffold. IPC fibers were incorporated in a circumferential orientation. (FIG. 30A) Cumulative release profile of lysozyme from PVA-IPC fiber composite planar film. (FIG. 30B) Cumulative release profile of lysozyme from PVA-IPC fiber composite tubular scaffold.

FIGS. 31A and 31B show controlled release of VEGF (44 kDa) from IPC fibers incorporated into PVA tubular scaffold and its effect on endothelial cell proliferation. IPC fibers were incorporated in a circumferential orientation. (FIG. 31A) Cumulative release profile of VEGF from PVA-IPC fiber composite tubular scaffold, compared to standalone IPC fibers with VEGF. (FIG. 31B) Bioactivity of released VEGF as measured through an endothelial cell proliferation assay.

FIGS. 32A and 32B show controlled release of PEG-QK (11 kDa) from IPC fibers incorporated into PVA tubular scaffold and its effect on endothelial cell proliferation. IPC fibers were incorporated in a bi-directional angular orientation. (FIG. 32A) Cumulative release profile of QK-PEG from PVA-IPC fiber composite tubular scaffold, compared to standalone IPC fibers with QK-PEG. (FIG. 32B) Bioactivity of released VEGF as measured through an endothelial cell proliferation assay.

FIGS. 33A, 33B, 33C and 33D show H&E and IHC staining of ischemic hind limb tissue (FIG. 33A, B) and tissue surrounding PVA graft (FIG. 33C, D) to detect macrophage infiltration. Brown staining denotes positive stain for Mac387, a monocyte/macrophage marker.

### DETAILED DESCRIPTION OF THE INVENTION

The present invention is directed to synthetic small diameter vascular grafts (SDVG) engineered to improve patency, cell attachment, and endothelialization. Topography is known to improve adhesion and proliferation of endothelial cells in vitro. Thus, topography aids endothelialization of synthetic vascular grafts and tissue remodeling, consequently improving functionality and long-term patency. Applicants employed both biophysical cues (topography) and biochemical cues (attachment factors) to improve the bioactivity of PVA scaffolds, demonstrating increased endothelial cell adhesion, retention of phenotype, and improved hemocompatibility.

In certain aspects, the invention relates to patterning of the PVA hydrogel through solvent casting, an economical method that avoids the use of extra expensive or advanced equipment often reserved for industrial use. Moreover, patterning of PVA using the solvent casting method avoids the use of other crosslinking chemicals that may have an undesired side effect when implanted in vivo. Importantly, the patterning of PVA using the solvent casting method yielded topographies with very high fidelity and integrity. Similarly, tubular PVA scaffolds with luminal surface topography are created through dip casting. However, this technique is limited to topographies in the micrometer range. The dip-casting process may be improved with the use of a tubular mold with the pattern etched on its hydrophilic surface. Additionally, the crosslinking of biochemical cues with PVA requires only the mixing of a small volume of the desired biochemical factor, thereby avoiding complex surface grafting mechanisms or multi-step modification processes.

### Poly(Vinyl Alcohol)

Previously, Chaouat demonstrated a novel method for PVA crosslinking, in which sodium trimetaphosphate (STMP), a non-toxic compound commonly used in the food industry, was added to the aqueous PVA solution in alkaline conditions [Chaouat M, et al., Advanced Functional Materials 18, 2855-286 (2008)1]. The resultant vascular graft exhibited excellent mechanical properties such as suture retention strength and compliance, better approximating to native arterial tissue compared to PTFE and Dacron. Grafts implanted into rat abdominal aortas showed patency rates of 83% at 1 week post-implantation, with no detectable aneurysm formation. As the cross-linking process did not involve toxic components and organic solvents, there was no problem of residual toxicity. However, the inherent hydrophilic nature of PVA discouraged cell attachment. Hence, PVA appears to make for a poor support frame for the formation of an endothelial monolayer that will ensure bioactivity and long term patency of the vascular graft.

Therefore, there exists a need for modifications of poly(vinyl alcohol) to promote its endothelialization after implantation. Spontaneous endothelialization occurs through any or all of the following processes: direct migration of endothelial cells from the anastomotic edge into the graft, transmural migration of endothelial cells or cell transformation from endothelial progenitor cells. Various biochemical cues, such as vascular endothelial growth factor, have been widely explored to modify luminal surfaces of grafts to increase adhesion of endothelial cells. On the other hand, surface topographical cues have not yet been explored for the purpose of improving vascular graft in situ endothelialization.

### Topographical Cues

Surface topographical cues have been employed to affect various cell behaviours. It is generally acknowledged that cells are able to sense the stiffness and contours of its underlying surface, and by mechanotransduction, the mechanical stimulus is converted into internal biochemical signalling pathways, which in turn affects cell behavioural patterns such as attachment, morphology, migration and proliferation. In endothelial cells, it was observed that surface porosity enhances endothelialization [Sarkar S., et al., Applied Biomaterials 82, 100-8 (2007); Ranjan A, Webster T J. Nanotechnology 20, 305102 (2009)], a preliminary indicator of the influence of surface roughness on endothelial cell behaviour. In terms of cell morphology, the observed alignment of endothelial cells to linear patterns, both on the micro- and nano-scale, has been well-characterized [Biela S. A., et al. Acta Biomater. 5, 2460-6 (2009); Uttayarat, P., et al., MRS Proceedings Volume 845, 2004 Fall Meeting Symposium AA, Nanoscale Materials Science in Biology and Medicine; Bettinger C. J., et al., Angew. Chem. Int. Ed. Eng., 48, 5406 (2009); Ranjan A, Webster T J. Nanotechnology 20, 305102 (2009); Uttayarat P, et al., Am J Physiol Heart Circ Physiol. 294, H1027-35 (2008)]. Using grating patterns of 200 nm depth and 2 μm groove width, Biela et al. demonstrated that human coronary artery endothelial cells not only displayed an elongated morphology along the longitudinal grating axis, but also had a tendency to migrate in the axis direction as well [Biela S. A., et al. Acta Biomater. 5, 2460-6 (2009)]. Similarly, Uttayarat et al. also showed that cells migrated overwhelmingly in the direction parallel to the longitudinal axis, maintaining a steady migration speed over the 4-hour course of observation. This phenomenon was enhanced in the presence of moderate to high flow [Uttayarat P, et al., Am J Physiol Heart Circ Physiol. 294, H1027-35 (2008)].

The extracellular matrix (ECM) provides not only structural support for the attachment of cells; it also provides a combination of biophysical and biochemical cues that will direct cell behavior to respond to the environment. Topographical cues are one example of biophysical cues to which cells have a physiologic response. The results presented herein show the improvement of endothelial cell viability, proliferation and function in response to gratings and microlens topography. Specifically, 2 μm gratings and the microlens structures were shown to improve PVA film bioactivity, possibly by mimicking the ECM ultrastructure of vascular endothelial cells. A previous study has observed a nanometer-sized, fibrous and porous ECM that are similar in geometry for all blood vessels regardless of its anatomical locations [Liliensiek S, et al., Tissue Eng Part A. 15, 2643-2651 (2009)]. Notably, the ECM fibrillar structure of the carotid artery showed increased alignment whereas the ECM of the aorta showed a more random alignment of fibers. This may be attributed to the difference in flow conditions between the anatomical locations. This fact is significant for SDVG, which are usually implanted in vessels with disrupted laminar flow, which is known to induce atherogenic phenotype in endothelial cells The use of topographical cues that are aligned along the grating axis may promote the anti-atherogenic phenotype of endothelial cells [Di Rienzo C, et al., Scientific Reports 3, 1141-1149 (2013)] by acting in contradiction to shear stress [Morgan J, et al., Biomaterials 33, 4126-4135 (2012)]. In some aspects of the present invention, the bioactive scaffold with topographical pattern is hemocompatible. Micro-sized gratings are useful for selective inhibition of smooth muscle cell proliferation that may prevent intimal hyperplasia.

Surface Modification with Attachment Factors

Biomaterials can also be modified with attachment factors to improve endothelial cell adhesion, for example through binding of integrin receptors. For example, Zheng et al. (2012) [Zheng W., et al., Biomaterials 33, 2880-2891 (2012)] incorporated RGD (the active tripeptide moiety of the ECM protein fibronectin) into the luminal surface of small diameter PCL grafts to improve functionality and patency in an animal model. They demonstrated faster endothelialization of RGD-PCL grafts after implantation in a rabbit carotid artery model. In comparison, bare PCL grafts were shown to have less endothelialization, as well as thrombosis in some instances.

Aside from the biophysical cue, biochemical cues were also introduced into the PVA hydrogel in the form of attachment factors. Fibronectin is a well-known ECM component that activates integrins through its RGD moiety, leading to cell adhesion. RGDS is a tetrapeptide derivative of fibronectin, while cRGD is a peptide derivative locked into a specific conformation. RGD-containing factors were shown to have improved endothelial cell adhesion, proliferation and function. On the other hand, heparin is an anticoagulant glycoprotein that was used for improving cell adhesion through a different mechanism: the enhancement of serum protein adsorption. Its effect on endothelial cell culture was not apparent, however. Possibly, this may be thru the difference in the mechanical property of the heparin-PVA scaffolds, which had less elasticity. It may also be due to complete immobilization of heparin in the PVA scaffold, preventing heparin from interacting with serum proteins. The multiple functional groups on the long chain of heparin may have caused an increase in crosslinking density. Moreover, heparin immobilization may have also led to a change in conformation, rendering it inactive.

In some embodiments of the invention, the bioactive scaffold is a topographically- and/or biochemically-modified film. Such films are useful as drug delivery vehicles, for applications such as tissue repair and regeneration as, for example, cartilage implants, as cardiac patches, for applications such as skin repair, wound healing, esophagus repair, repair of defects in the gastrointestinal tract, for example a colonic defect, repair of abdomen wall, or films may be used as reinforcing materials for repair of soft tissues such as tendons.

### Vascular Endothelial Growth Factor (VEGF) and its Use in Therapeutic Angiogenesis

Various growth factors, when applied around sites of occlusion, have been demonstrated to promote collateral vessel development and are regarded as promising for the treatment of occlusive vascular disease. Aside from the use of vascular endothelial growth factor (VEGF)-A as a survival, mitogenic and chemotactic factor for endothelial cells, VEGF-A is a key stimulator of angiogenesis [Ferrara, N., EXS 94, 209-31 (2005)]. The binding of VEGF-A to VEGF receptors has been demonstrated to promote new blood vessel formation [Ferrara, N., EXS 94, 209-31 (2005)]. It is required for both physiological and pathological angiogenesis.

However, randomized controlled clinical trials using VEGF and fibroblast growth factor (FGF) delivered as recombinant protein or via gene transfer for the treatment of peripheral ischemia have not shown convincing efficacy. It appears that the effects of VEGF-A are strongly dependent on its local concentration [Yla-Herttuala, S., et al., J Am Coll Cardiol, 49(10), 1015-26 (2007)]. Successful therapeutic angiogenesis requires prolonged VEGF expression for activation of endothelial and hematopoietic progenitor cells and maintenance of stable neovessels [Sun, Q., et al., Pharm Res 22(7), 1110-6 (2005)]. Another difficulty in the use of recombinant growth factors lies in their low accumulation in the ischemic tissue after intra-arterial administration and their rapid inactivation in vivo. There is a need to develop an efficacious delivery system to facilitate the success of growth factor therapy for peripheral arterial disease (PAD).

### Controlled Release of Growth Factors for Collateral Angiogenesis and Arteriogenesis

Therefore, the success of revascularization depends on the local concentration and a prolonged expression period, a sustained release of the growth factor could enhance the collateral angiogenesis for revascularization of the ischemic tissue and hence improve the treatment of PAD [Hosaka, A., et al., Circulation 110(21), 3322-8 (2004)]. Our previous studies have demonstrated that growth factors can be encapsulated and sustained released from the Interfacial polyelectrolyte complexation (IPC) fiber system. IPC is a self-assembly fiber formation process that makes use of electrostatic interactions between two oppositely-charged polyelectrolytes at physiological conditions. Biologics such as growth factors can be added to the polyanion/polycation component prior to complexation and be encapsulated into the fiber. It has been previously demonstrated that IPC fibers were able to sustainably release TGF-β3 [Yim, E. K., et al., Tissue Eng 13(2), 423-33 (2007)] and platelet derived growth factor (PDGF) [Liao, I. C., et al., J Control Release 104(2), 347-58 (2005)] over a period up to 27 days with retained bioactivity.

Therefore, the present invention is directed to the utilization of a combination of biochemical and topographical cues to improve the bioactivity and performance of PVA-based vascular grafts. The incorporation of surface topographical cues, attachment factors and endothelial biochemical cue in a PVA based vascular graft aims to accelerate the formation of a confluent endothelial monolayer, thus ultimately improving the long term patency of poly(vinyl alcohol) based grafts. The present invention relates to a method of producing a bioactive PVA-based scaffold for biological application such as vascular graft through: patterning of planar PVA by casting, patterning of tubular scaffold luminal surface by dip-casting, immobilization of attachment factors to enhance endothelial cell attachment and survival, and incorporation of bioactive growth factor or peptide into the PVA-based tubular scaffold.

The proof-of-concept in vivo tests demonstrated the biocompatibility, blood compatibility and patency of the PVA-based in a small-diameter vascular graft application.

The present invention allows for a number of advantages over the prior art, including a novel PVA chemical crosslinking method, which is performed in aqueous solution and does not involve organic solvent or toxic component. This PVA cross-linking method may occur in the presence of IPC fiber or an adhesion factor, components which may interfere with the cross-linking process. The present invention allows for uniform modification of the surface of the PVA scaffold without the use of chemical linker, resulting in covalent binding of the adhesion factor on the scaffold surface. The invention is further advantageous in that the resulting vascular grafts possess excellent and easily tunable mechanical properties and retain hemocompatibility. Further still, surface topography and modification with attachment factors improve the attachment, survival and migration of endothelial cells from the native arteries into the vascular grafts, and therefore improve the endothelialization of the vascular grafts. Finally, the grafts of the present invention allow for sustained delivery of biochemical cues for stimulating endothelial cell function and possibly, therapeutic angiogenesis.

Traditionally, chemical crosslinking of PVA is performed with the use of glutaraldehyde [Peppas N A, Merrill E W. J. Biomed. Mater. Res 11(3), 423-34 (1977)]. Any residual crosslinker will be hazardous in vivo when glutaraldehyde-PVA crosslinker is used as a biomedical implant. Other methods of crosslinking PVA use freeze-drying methods [Holloway J L, et al., Acta Biomaterialia 7(6): 2477-82 (2011)] or ultraviolet light for crosslinking [Bourke S L, et al., AAPS PharmSci 5(4), 101-11 (2003)]. Both methods are better than glutaraldehyde-PVA for in vivo implantation. However, these two methods may not allow very precise control of crosslinking and resulting mechanical properties without costly machines that provide accurate and well-controlled conditions for freeze-drying and UV light emission. On the other hand, the use of STMP and NaOH for chemically crosslinking PVA have several advantages: first, they require chemicals that are non-hazardous, easy to prepare and easy to obtain, thus making PVA scaffold fabrication economical; second, STMP is a food-grade crosslinker that is biocompatible; third, fabrication of PVA scaffolds with different mechanical and chemical properties is facile and flexible.

The criteria for graft mechanical properties are stringent. Mechanical characteristics of grafts such as compliance, Young's modulus and size have considerable potential in determining graft patency [Salacinski H. J., et al., Journal of Biomaterial Applications 15, 241-78 (2001)]. Graft compliance is a widely-acknowledged benchmark for graft patency, as compliance mismatch between the graft and vessel wall may contribute to intimal hypertrophy, turbulent blood flow and impedance mismatch or disrupted wall shear stress [Stewart S F, Lyman D J, Journal of Biomechanics 23, 629-37 (1990)]. In this respect, materials such as Dacron and PTFE, with Young's modulus of 14 GPa [Salacinski H. J., et al., Journal of Biomaterial Applications 15, 241-78 (2001)] and 0.5 GPa [Salacinski H. J., et al., Journal of Biomaterial Applications 15, 241-78 (2001)] respectively, face graft patency problems as they are stiffer than native arteries, which are estimated to be around 0.4 MPa [Di Rienzo C, et al., Scientific Reports 3, 1141-1149 (2013); Morgan J, et al., Biomaterials 33, 4126-4135 (2012)]. On the other hand, PVA has been shown previously by Chaouat et al, to have mechanical properties near the physiological range. For example, its Young's modulus is reported at 0.2 MPa, which is closer to the physiological level than either Dacron or PTFE [Chaouat M, et al., Advanced Functional Materials 18, 2855-2861 (2008)].

Synthetic materials available in the market today still pose problems of having inherent thrombogenic properties. To improve on the thrombogenicity of these materials, studies have been done to seed native cells directly on to scaffolds to mimic native vessel constituents. However, this method is tedious, time consuming and is not easy to be scaled up for industrial production. Moreover, this requires the additional step of harvesting autologous cells; that is, cells that come from the patient who will be receiving the graft. This step can be replaced by the use of the surface topographical cues and attachment factors, which will initiate endothelialization of the PVA vascular graft after implantation.

The vascular graft is also equipped with the capability to be a device for sustained delivery of therapeutic protein or growth factor. It is commonly observed that lower limb ischemia in patients, especially those with diabetes, are caused by both macro- and micro-occlusion. A treatment such as a device (i.e. vascular graft) with controlled release of therapeutic/angiogenic protein that targets both levels of obstruction could enhance the recovery. A PVA vascular graft can cure macro-occlusion through replacement or bypass of an occluded major artery and maintenance of patency through endothelialization (as discussed in point b). Meanwhile, micro-occlusion (local or systemic) can be treated through the sustained and controlled release of angiogenic factor such as QK-PEG.

### EXAMPLES

### General Methods

### Endothelial Cell Culture

HUVEC (Lonza) were grown in EGM-2MV growth medium (Lonza) at standard cell culture conditions of 37° C. and 5% CO₂. HUVEC were washed with HEPES buffered saline solution and trypsinized using 0.05% Trypsin to harvest cells. Subsequently, cell count was performed on viable cells stained with trypan blue. A total of 200,000 cells/cm² in 500 μl EGM-2MV medium was added to each PVA film. The cells were stimulated to adhere to the PVA films by centrifugation at 220×g for 5 minutes at 25° C. Seeded PVA films were incubated in standard culture conditions for 24 hours. All patterned and the unpatterned PVA film was used in triplicate for various cell seeding experiments.

### EdU and Live/Dead Assay

To assess the proliferative capacity of HUVEC on PVA films, the Click-iT EdU assay kit (Life Technologies) was used. EdU is a thymidine analog that will be incorporated into mitotic cells. Fluorescent detection of EdU allows quantification of the number of proliferating cells, in comparison to the total number of cells. At 20 hours post-seeding, EdU was added to each PVA sample. PVA samples were thereafter fixed and stained for EdU and DNA, according to the manufacturer's instructions. Similarly, cell viability was assessed by using the Live/Dead assay kit (Life Technologies).

### Immunofluorescence

Assessment of HUVEC functionality and morphology after seeding on PVA films was performed using immunofluorescence staining. Twenty four hours post-seeding, PVA films were fixed for 15 minutes using 4% paraformaldehyde in phosphate buffered saline (PBS). Permeabilization of cells (if necessary) was performed using 0.1% Triton X-100 in PBS for 15 minutes, while blocking of the films was performed using 2% BSA in PBS for 1 hour at room temperature. Thereafter, primary antibodies against various cell surface (CD31, VE-Cadherin) and intracellular (vWF, eNOS, phosphorylated FAK [pFAK]) antigens were incubated with the PVA films overnight. Secondary antibodies were then added for 1 hour at room temperature. Nucleus was visualized using DAPI, and actin fibers were visualized using fluorescently-labelled phalloidin.

### Matrigel Assay

To assess the in vitro angiogenic capacity of HUVEC on PVA planar films, a Matrigel assay was employed. Briefly, 50 μl of Matrigel (BD Bioscience) was placed in each well of a 96 well plate and allowed to gel at 37° C. for at least one hour. PVA films were moved to a new 24 well plate using sterile forceps 24 hours post-seeding. PVA films were then washed with HEPES buffered saline solution and cells were harvested using 0.05% trypsin. Cells grown on the same type of PVA film were pooled together and counted using the trypan blue viability stain. Thereafter, 15000 cells in 100 μl of EGM-2MV medium were placed in Matrigel. Tube formation was assessed by light microscopy after 8 hours.

Incubation with Platelet Rich Plasma (PRP) and SEM Visualization

In vitro blood compatibility assay was performed according to Yim et al [Tissue Eng 13(2), 423-33 (2007)]. Blood samples were collected from from healthy, male, New Zealand White rabbits in polypropylene tubes primed with heparin (5 U per ml blood). Blood was centrifuged at 100 g for 12 minutes at 22° C. PRP was then collected and placed in a separate polypropylene tube. PVA scaffolds were sterilized with a penicillin-streptomycin solution and UV sterilized for 30 minutes. PVA films were then washed extensively with sterile DI H₂O extensively before incubation with PRP. Silastic tubing (Dow corning) was used to anchor PVA films (1 cm×1 cm) on to the bottom of a well in a 24 well plate. PRP was then added to each sample at 200 μl per mg sample and sealed at both ends with Luer Lock Combi-stoppers (BBraun). Glass beads coated with incubated for 1 hour at 37° C. while rotating at 100 rpm. Resting samples of 100 μl PRP alone and 100 μl PRP with anti-platelet disodium EDTA (5.4 mM) were also included in the experiment. Thereafter, PRP was collected for subsequent flow cytometry analysis [0090], while PVA scaffolds were kept for platelet morphology analysis using SEM. Samples were washed in phosphate buffered saline and fixed using 2.5% glutaraldehyde at 4° C. for 2 hours. Thereafter, films were dehydrated using a series of increasing ethanol concentration. After complete drying, films were then coated and visualized under SEM, as described in [0094]. Assay was done in triplicate for each sample.

### Flow Cytometric Analysis of Platelet Activation

After incubation, PRP was collected and placed in 1.5 ml polypropylene tubes. Aliquots of PRP (10 μl) were diluted with 200 μl of HEPES-Tyrodes buffer (HTB; 137 mM sodium chloride, 2.7 mM potassium chloride, 16 mM sodium bicarbonate, 5 mM magnesium chloride, 3.5 mM HEPES, 1% glucose, 2% bovine serum albumin, pH 7.4) and incubated with antibodies against CD61/GPIIb/IIIa (Abbiotec). Platelets were then washed with HTB and centrifuged at 21000 rpm for 5 minutes at 4° C. Platelets were then resuspended in 100 μl HTB and incubated with fluorescently-conjugated secondary antibodies. Samples were fixed with 2% paraformaldehyde in HTB. Platelets were analyzed within 24 hours for scatter characteristics and expression of markers using BD LSR Fortessa. Flow cytometry data was analyzed using FlowJo 4.0.

### Activation of PVA for Crosslinking

An aqueous PVA solution of 10% (w/v) was prepared by dissolving PVA (85-124 kDa; 87-89% hydrolyzed) in distilled deionized (DDI) water. The solution was autoclaved at 121° C. and mixed overnight until homogenization of the solution. PVA solution was stored at 4° C. until future use. To activate PVA for crosslinking, the crosslinker STMP, was freshly prepared in DI water at a concentration of 15% (w/v) and added to PVA solution. For every 12 g of PVA, 1000 μl of 15% STMP (w/v) was added. The solution was stirred for 5 minutes to ensure homogeneity. Crosslinking by STMP was activated by the dropwise addition of 400 μl of 30% (v/v) sodium hydroxide for every 12 g of PVA. The mixture was centrifuged at high speed (1000-2000 rpm) for 15 minutes to remove bubbles. The mixture, heretofore referred to as the “activated PVA solution” was then used immediately for both fabrication of planar patterned PVA film and tubular PVA film with pattern on the luminal surface.

Unpatterned small diameter PVA graft was fabricated and implanted in the rabbit femoral artery. Table 1 shows that the mechanical properties of 0.9 mm PVA small diameter vascular grafts closely approximated that of the rabbit femoral artery.

TABLE 1
* denotes statistical significance between 1 mm diameter PVA graft and rabbit femoral artery.

| | Internal | Wall | | Burst | Suture | |
|---|---|---|---|---|---|---|
| | diameter | thickness | Compliance | pressure | retention | Young's |
| Tube | (μm) | (μm) | (%) | (mmHg) | (g) | modulus (kPa) |
| 0.9 mm | 1092 ± 83.77 * | 310.7 ± 51.08 | 4.793 ± 3.252 | 867.8 ± 171.1 | 95.63 ± 2.803 | 833.1 ± 76.44 |
| diameter | | | | | | |
| PVA graft | | | | | | |
| Rabbit | 1999 ± 330 | 350-710 | 5.9 ± 0.5 | 2031-4225 | 200 ± 119 | 230¹, 11000² |
| femoral | | | | | | |
| artery^{1,2} | | | | | | |

¹Demaria 2000 gp;
²Uchida 1989 uj.
* denotes statistical significance between 1 mm diameter PVA graft and rabbit femoral artery.

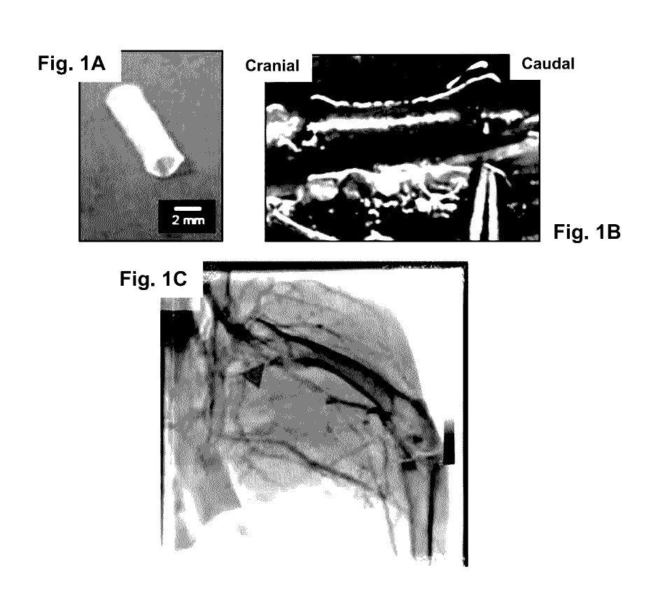

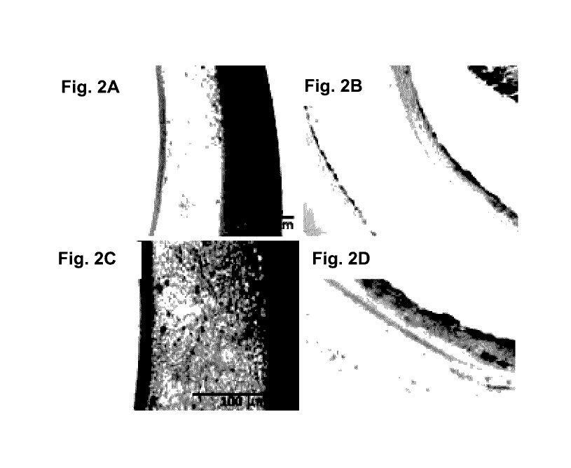

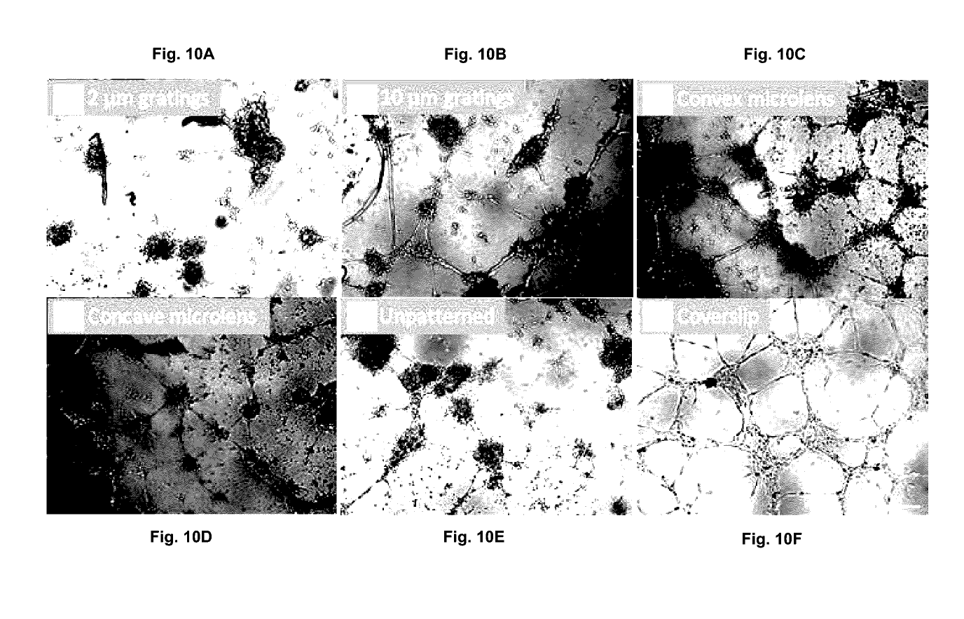

FIGS. 1A, 1B and 1C show representative images of PVA small diameter vascular grafts, isolated (FIG. 1A) and implanted in the rabbit femoral artery (FIG. 1B). In total, three PVA small diameter vascular grafts with internal diameter of either 0.9 mm or 1 mm (with the choice of caliber depending on the size of the native artery) were anastomosed to the left femoral artery of a rabbit PAD model. PAD was induced through the embolization and partial occlusion of the digital artery (major artery supplying the foot). At approximately two weeks, 2 out of 3 PVA small diameter vascular grafts remained patent, as assessed by digital subtraction angiography (FIG. 10). Immunohistochemistry analysis of explanted PVA small diameter vascular grafts (FIGS. 2A, 2B, 2C and 2D) also showed endothelial cells on the luminal surface of the patent PVA vascular graft (FIG. 2B, FIG. 2D). Our preliminary animal studies demonstrate the tunable mechanical properties, suturability, implantability and short-term patency of the PVA graft with a sub-millimeter diameter.

Fabrication of the Planar PVA Film with Topography Via Casting

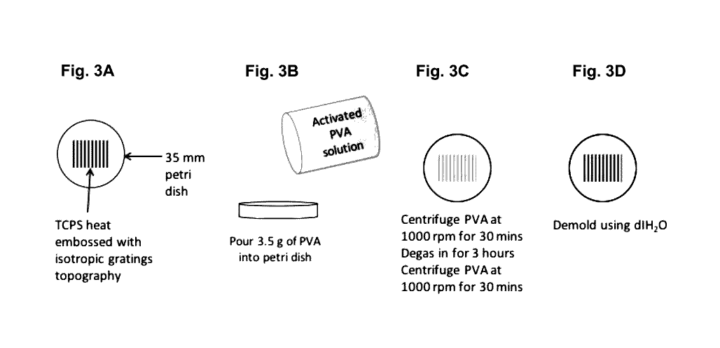

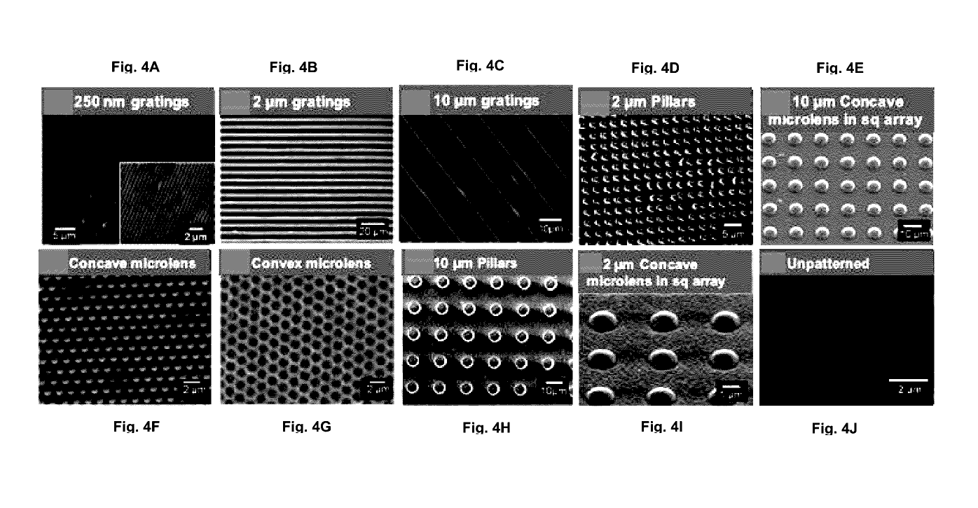

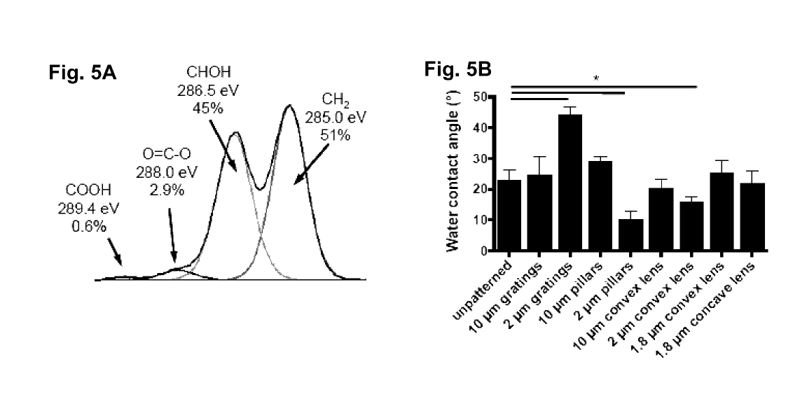

FIGS. 3A-D show the schematic of casting to create a planar PVA film with surface topography. Patterned planar PVA substrates were used for in vitro tests that determine the effect of topographical cues on the bioactivity of PVA. (FIG. 3A) A piece of tissue culture polystyrene (TOPS) was patterned using heat embossing. Afterwards, the TOPS template was washed with 0.01% Triton-X 100 in distilled deionized water and dried with nitrogen air. Alternatively, polydimethylsiloxane (PDMS) molds with the desired patterns can be used as templates for patterning PVA. PDMS molds were modified with oxygen gas (9 L/min) plasma at 85 watts, 1 min. Activated PVA solution (3.5 g) was poured on top of the TOPS or PDMS mold (FIG. 3B). To allow PVA to flow into the mold patterns, the PVA was centrifuged on the mold for 30 minutes at 1000 rpm (FIG. 3C). PVA was then degassed for 3 hours before centrifugation for an additional 30 minutes at 1000 rpm. The activated PVA solution was then allowed to be crosslinked very slowly, for at least 5 days at 18° C. (FIG. 3D). The use of a very slow drying process is critical to the patterning of PVA. FIGS. 4A-J show scanning electron microscope (SEM) images of the planar film with topography. Anisotropic patterns such as gratings (ridge×gap×height of 250 nm×250 nm×250 nm; 2 μm×2 μm×2 μm; 10 μm×10 μm×10 μm) and isotropic patterns such as microlens (concave and convex, diameter 1.8 μm, pitch 2 μm, sag 0.7 μm) or pillars (diameter×height 10 μm×10 μm; 2 μm×2 μm) were replicated successfully with excellent fidelity on PVA planar films (FIGS. 4A-4J). Planar patterned PVA films were immersed in sterile DI H₂O and allowed to swell before demolding. XPS analysis of the surface of unpatterned PVA showed characteristic carboxyl, hydroxyl, methyl and carbonyl groups (FIG. 5A). Water contact angle analysis of PVA with various topographies show changes when compared with unpatterned PVA (FIG. 5B).

### Scanning Electron Microscopy (SEM) Analysis of PVA Film

To prepare for SEM analysis, PVA films were air-dried overnight at ambient temperature. Dry PVA samples were then coated (JEOL-JFC 1600 auto-fine coater) with 10 nm platinum film. Topographical features of PVA tube were visualized using JEOL-JSM 6010LV scanning electron microscope at high vacuum and 10 kV.

### Effect of Surface Patterned PVA Films on Cell Alignment, Morphology and Function

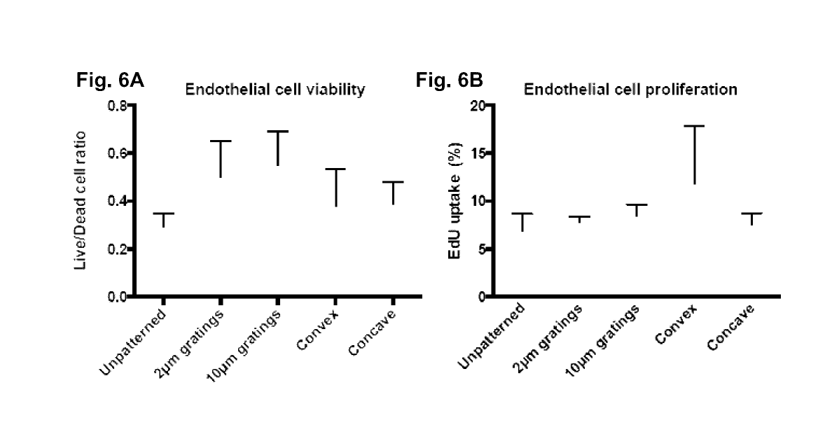

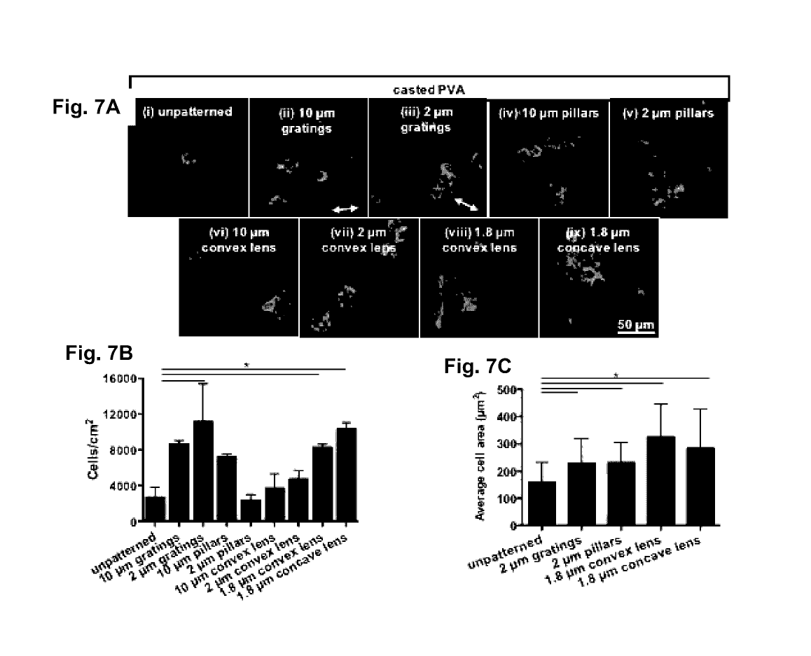

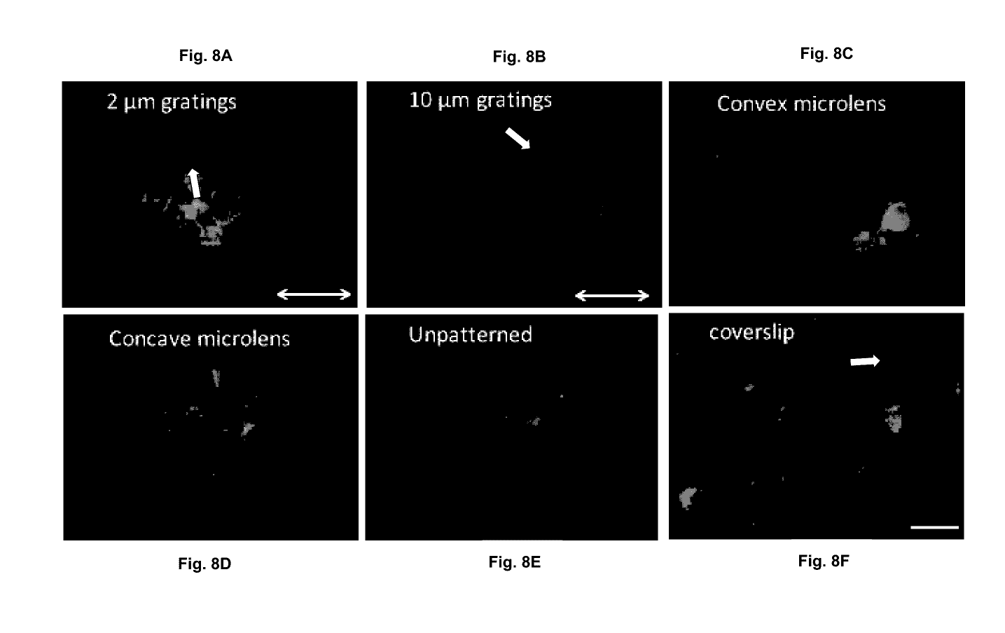

We tested the effect of the 2 μm and 10 μm anisotropic gratings and the convex and concave microlens patterns on PVA on endothelial cell viability, phenotype and functionality after 24 hours. For comparison, endothelial cells were also cultured on unpatterned PVA films. FIG. 6A shows the viability of endothelial cells after 24 hours of culture on various patterned PVA films as measured by Live/Dead assay (Life Technologies). Introduction of topography increased the viability of endothelial cells, in contrast to those grown on unpatterned PVA films. In addition to cell viability and proliferation (FIG. 6B), adhesion and area of human endothelial cells were shown to improve on selected planar PVA with topography (FIGS. 7A-7C). Furthermore, endothelial cells on patterned PVA films show better retention of endothelial cell phenotype (FIGS. 8A-8F). Endothelial cells show positive staining for CD31, an endothelial cell marker, after culture on patterned PVA films. In contrast, endothelial cells on unpatterned PVA show a lower level of CD31 expression.

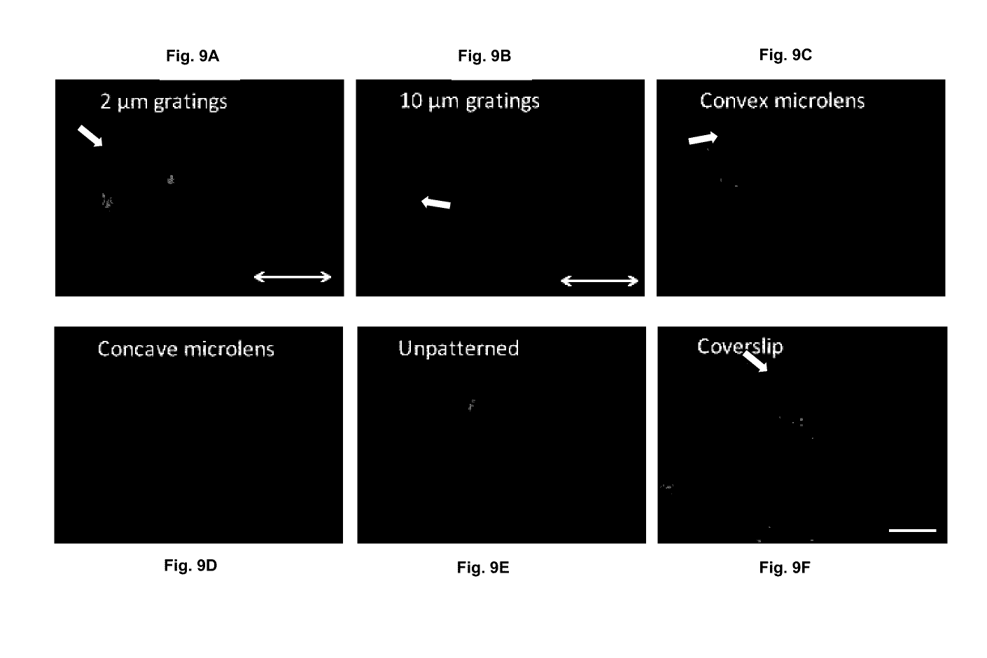

Concomitantly, the cells grown on patterned PVA films were shown to retain endothelial cell function, as marked by the presence of von Willebrand factor (vWF) (FIGS. 9A-9F). Endothelial cells on convex microlens and 2 μm gratings PVA films showed better retention of vWF compared to other patterned PVA films. More significantly, cells from 10 μm gratings and convex microlens PVA films, when harvested and reseeded on Matrigel, showed tubular formation, denoting the in vitro angiogenic capacity of these cells (FIGS. 10A-10F). Specifically, cells on convex microlens (FIG. 100) show the best tubular structure formation. Endothelial cells on concave microlens (FIG. 10D) and 10 μm gratings (FIG. 10B) also show some tube formation. On the other hand, cells harvested from unpatterned control (FIG. 10E) and 2 μm gratings (FIG. 10A) on PVA lack interconnected tubes and most of the cells are shown in clumps, demonstrating that the cells did not retain angiogenic capacity. Overall, the microlens structure and the 2 μm gratings structure seem to be the most beneficial for endothelial cell growth, proliferation and retention of function. Overall, PVA with topographical cues help to retain endothelial cell viability and retain endothelial function.

Fabrication of the PVA Vascular Graft with Luminal Surface Topography

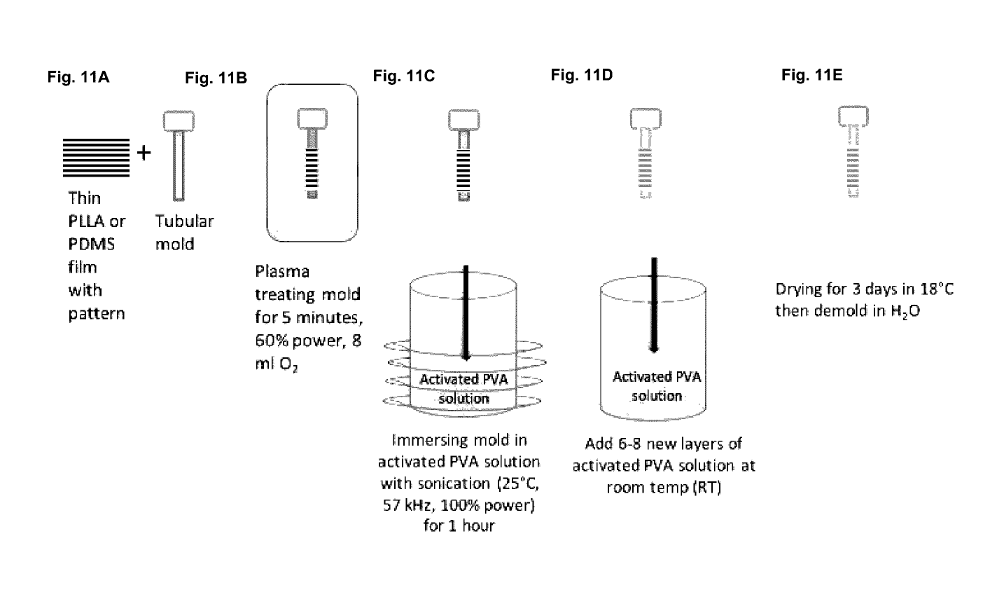

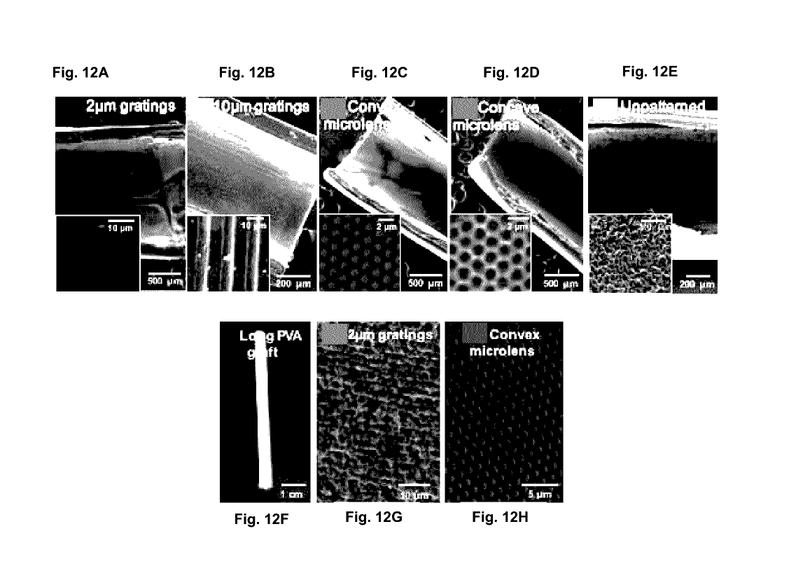

FIGS. 11A-E show the schematic of dip-casting to create tubular PVA graft with luminal topography. To create the PVA graft with luminal surface topography, a thin film of micropatterned polydimethylsiloxane (PDMS) or patterned poly-L-Lactic Acid (PLLA) or polymer that is not water soluble and stable in high pH, was wrapped and secured around a rod with a uniform outer diameter; this will serve as the tubular mold for the vascular graft (FIG. 11A). A hypodermic needle with outer diameter 0.9 mm and carbon rod with outer diameter 1 mm were used as a template for the fabrication of PVA tubular scaffolds. The PDMS or PLLA film was secured onto the rod in such a way that the patterned surface was outward and there are no gaps in between the PDMS film along the longitudinal axis of the graft. This setup will be further referred to as the “mold”. The molds used for patterning the PVA vascular graft contained grating topographies or microlens topographies (as used for the patterning of planar PVA film). To promote the movement of activated PVA solution into the topographical pattern on the PDMS or PLLA film, the mold was plasma treated with oxygen for 1 minute at 60 W (FIG. 11B). Consequently, the mold was dipped into the activated PVA solution and centrifuged at 1000 rpm for 30 minutes at 25° C. To further enhance the movement of the PVA into the topographical pattern, the mold with the activated PVA solution was then sonicated at 59 kHz for 1 hour at 20° C. in a water bath (FIG. 11C). Subsequently, the mold was centrifuged with the activated PVA solution at 1000 rpm for 1 minute at 25° C. to ensure the removal of microbubbles. Afterwards, the molds were removed from the activated solution and allowed to stand for 10 minutes. To improve the mechanical properties of the vascular graft, the mold was then dipped into a freshly prepared, activated PVA solution to add layers of PVA on to the graft (FIG. 11D). The PVA layer was allowed to dry for 15 minutes at ambient temperature before continuing with the next dip-casting. The dip-casting method was repeated for 6 to 8 more times, depending on the desired mechanical property of the graft. Tubular scaffolds were allowed to dry at 20° C. for 3 days. Scaffolds were washed several times in phosphate buffered saline (PBS) and allowed to swell in DI H₂O before removing from the tubular mold (FIG. 11E). FIGS. 12A-12E show SEM pictures of short (approximately 1 cm length), small diameter (2.25 mm internal diameter) PVA grafts with luminal topography. Prototypes of long (approximately 7 cm length) PVA grafts with luminal topography was demonstrated for 2 μm gratings and convex microlens (FIGS. 12F-12H).

### In Vivo Validation of Patterned PVA Vascular Graft in Rat Model

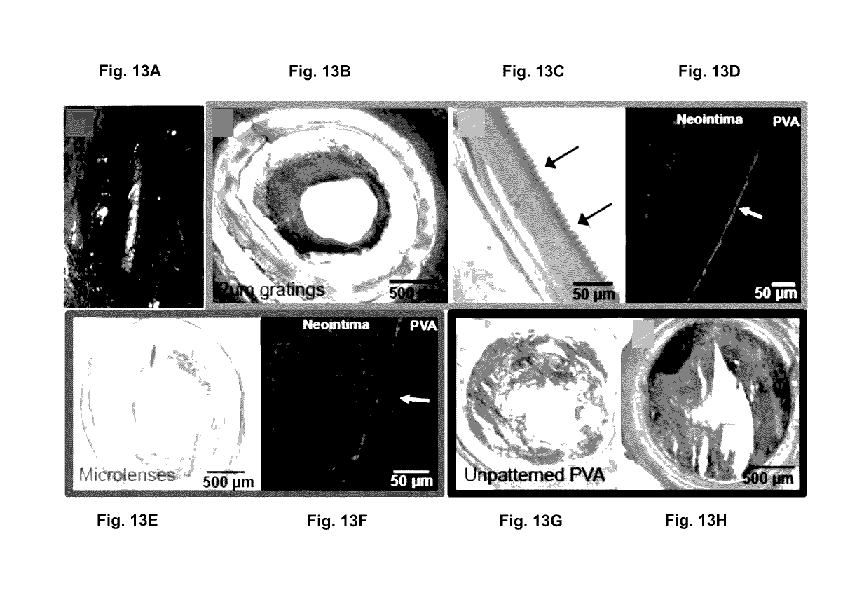

Collaborations in France showed positive results in the implantation of PVA vascular graft with surface topography (2 μm gratings, convex microlens and cRGD) in a rat abdominal aorta model (FIGS. 13A-H). PVA vascular grafts (2 mm diameter) modified with either 2 μm gratings (n=2) or both cRGD and convex microlens (n=1) were implanted (FIG. 13A). Subjects implanted with bioactive PVA grafts remained patent for 20 days (FIGS. 13B, E). Moreover, 2 μm gratings was still visible after 20 days (FIG. 13C), thus affirming robust patterning of the PVA vascular graft. Most importantly, staining against RECA antigen demonstrated endothelial cell attachment on gratings (FIG. 13D) or microlenses on cRGD-PVA (FIG. 13F). In contrast, PVA grafts without any pattern or cRGD modification were not patent. PVA grafts with either 2 μm gratings and cRGD modification or microlens were also occluded. Our preliminary results reiterate the benefit of microtopography in endothelialization of PVA small diameter vascular grafts.

Fabrication of PVA Modified with Biochemical Cues for Cell Attachment

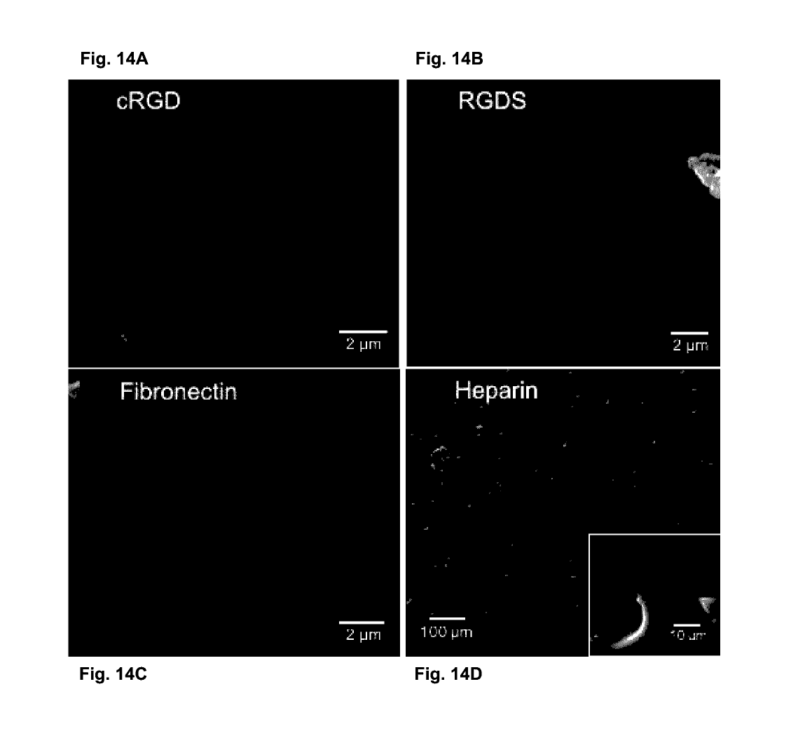

To further enhance the endothelialization on PVA, PVA was crosslinked with various attachment factors including heparin, fibronectin, RGDS tetrapeptide and cyclic RGD (cRGD) molecules. The novel modification was achieved simply by mixing the heparin (142.86 U per mg PVA) fibronectin (28.57 mg per mg PVA), RGDS (28.57 μg per mg PVA) or cRGD (28.57 μg per mg PVA) with activated PVA solution. The modified surfaces (FIGS. 14A-D) also showed minimal surface roughness that is comparable to that of unpatterned PVA, except for that of heparin-modified PVA, which appears to have a rough surface on the film, similar to microbubbles.

PVA films modified with biochemical cues were dried in ambient temperature. Thereafter, surface analysis of biochemically modified PVA was performed using X-ray photoelectron spectroscopy (XPS). A standard aluminum X-ray source (1486.7 eV, 75 W) was used to determine survey and high-resolution spectra. Static water contact angle measurement was also performed on the dried modified PVA films. One microliter of distilled deionized water was deposited on the PVA surface. The contact angle was measured after stabilization of the water droplet on the surface (Table 2).

TABLE 2

| | Elemental analysis‡ | | | | | Water |
|---|---|---|---|---|---|---|
| PVA | N | O | C | O/C | N/C | contact |
| modification | (%) | (%) | (%) | ratio | ratio | angle (°) |
| Unmodified | ND | 30.4 | 69.6 | 0.44 | ND | 25.9 ± 1.87^{D} |
| cRGD | 0.76 | 29.3 | 68.8 | 0.43 | 0.01 | 53.6 ± 0.69^{AB} |
| Fibronectin | 0.70 | 30.3 | 69.0 | 0.44 | 0.01 | 40.5 ± 1.96^{C} |
| Heparin | 0.78 | 32.3 | 66.9 | 0.48 | 0.01 | 53.7 ± 3.12^{A} |

Water contact angle data that are statistically different are separated by different letters.
ND = non detectable.
‡The balance in XPS for untreated and plasma-treated PVA films were Si, P. Statistically different data are separated by different letters.

PVA films modified with the various biochemical factors (fibronectin, cRGD peptide, RGDS peptide and heparin) showed a change in elemental composition compared to Unmodified PVA (Table 2). High resolution XPS analysis showed that nitrogen contributed by the biomolecules were detected on all modified films, and the N/C ratio were comparable between all modified PVA films. Water contact angle analysis showed that the wettability of the modified films significantly increased from the Unmodified PVA.

### Effect of Modified PVA Films on Cell Alignment, Morphology and Function

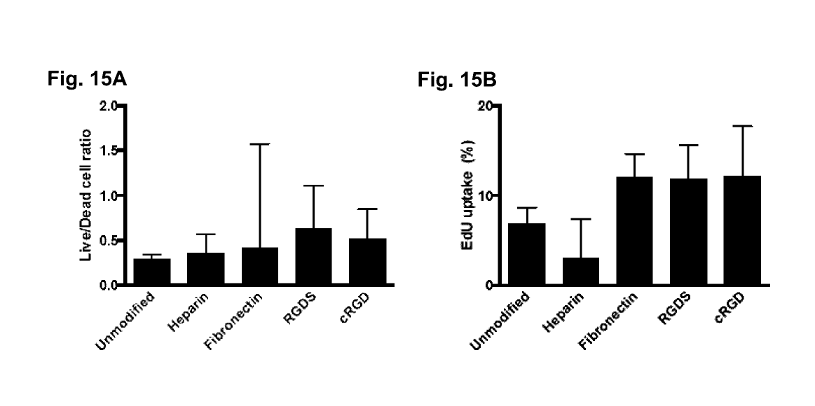

Endothelial cells were also cultured on modified PVA films. Live/Dead assay shows that the incorporation of RGDS and cRGD with PVA improved endothelial cell viability and endothelial cell proliferation (FIGS. 15A, 15B). On the other hand, heparin and fibronectin did not show any significant difference in cell viability compared to unpatterned PVA.

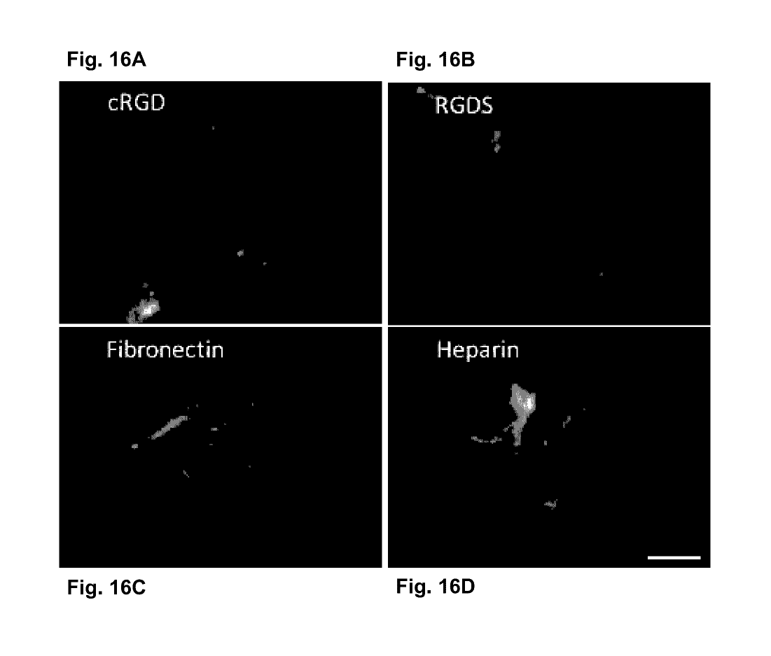

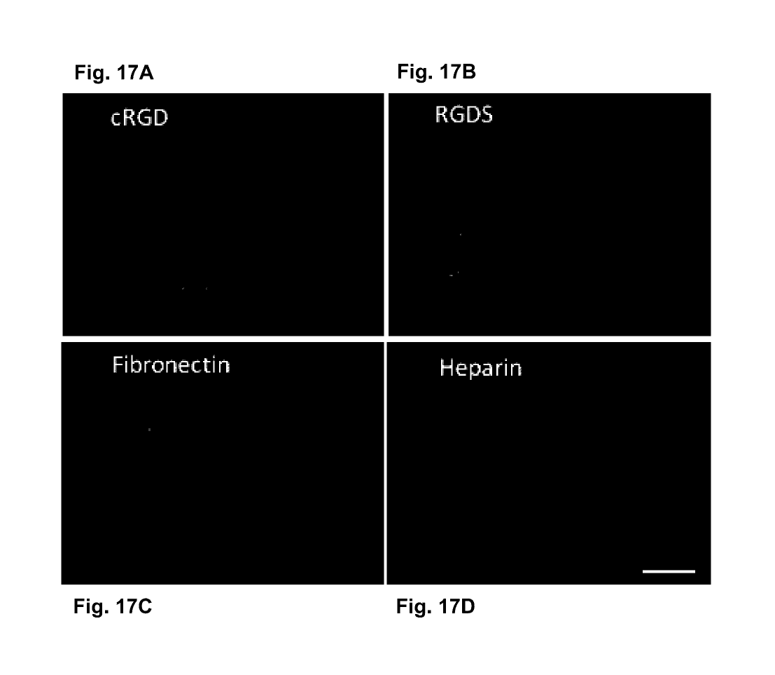

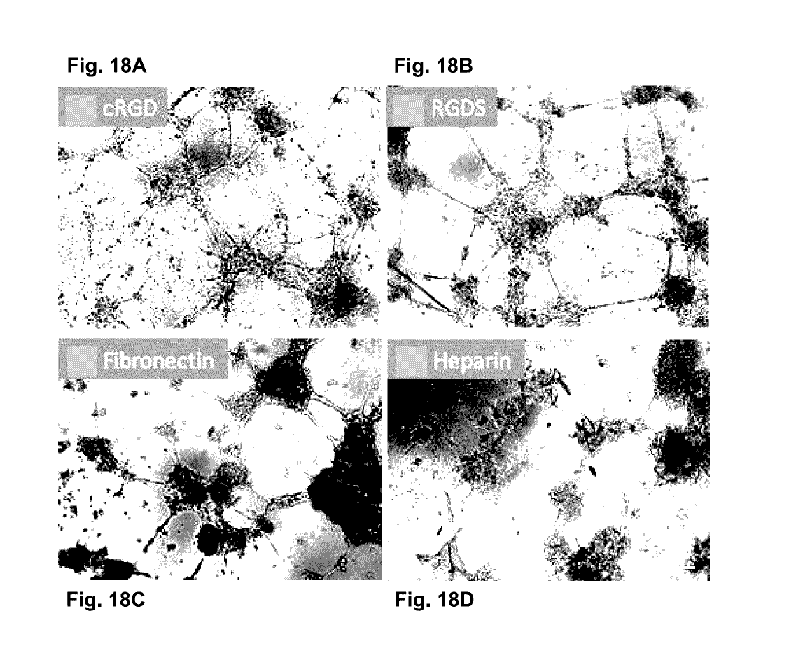

The endothelial cells grown on these modified PVA scaffolds also show retention of endothelial phenotype, as shown by immunofluorescence staining for CD31 (FIGS. 16A-16D). Endothelial cells also exhibited retention of vWF, especially for cells on RGDS and cRGD scaffolds (FIGS. 17A-17D). Matrigel assay shows that endothelial cells grown on the PVA scaffolds modified by fibronectin, RGDS and cRGD retain their angiogenic capacity, and in the case of RGDS-PVA show very good interconnected and regular tubular structures (FIGS. 18A-18D). On the other hand, cells grown on heparin modified PVA did not retain angiogenic capacity, as demonstrated by the lack of interconnected tube structures. Overall, modified PVA scaffolds improve endothelial cell viability and function.

Fabrication of PVA Films with Both Topographical and Attachment Cues

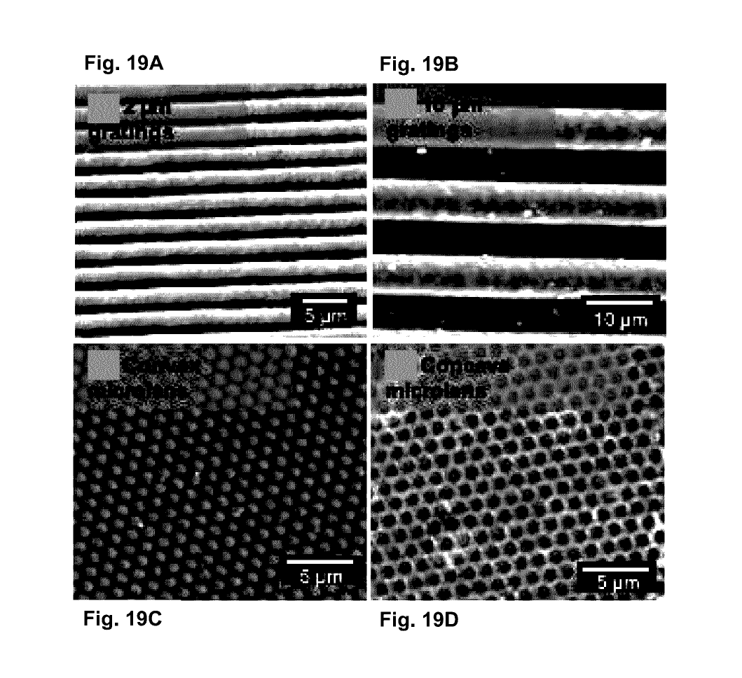

To synergistically improve endothelialization of PVA, both topographical and attachment cues can be employed on one PVA scaffold. Patterning of PVA was performed using the casting method, as described above, using activated PVA mixed with cRGD (instead of pristine PVA). FIGS. 19A-19D show the successful patterning of cRGD-PVA with both anisotropic patterns (2 μm×2 μm×2 μm, 10 μm×10 μm×10 μm gratings) and isotropic patterns (1.8 μm diameter, 2 μm pitch, 0.7 μm sag convex and concave microlens). Topographical cues on cRGD-PVA were replicated with good integrity.

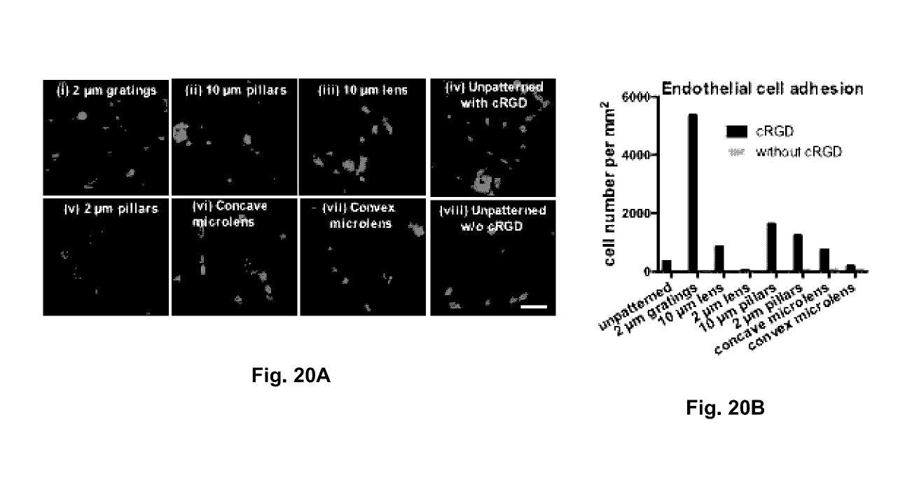

FIGS. 20A-20B show that with the addition of cRGD, cell attachment was significantly improved on unpatterned PVA after 24 hours of growth. The addition of topography was shown to substantially increase endothelial cell adhesion on PVA-cRGD over unpatterned cRGD substrate (FIG. 20A). The synergistic effect of the cRGD adhesion peptide and the topographical cues further improved PVA functionality and capability to support endothelial cell adhesion. This was most apparent with the notable increase of endothelial cell attachment on cRGD-PVA with 2 μm gratings (FIG. 20B).

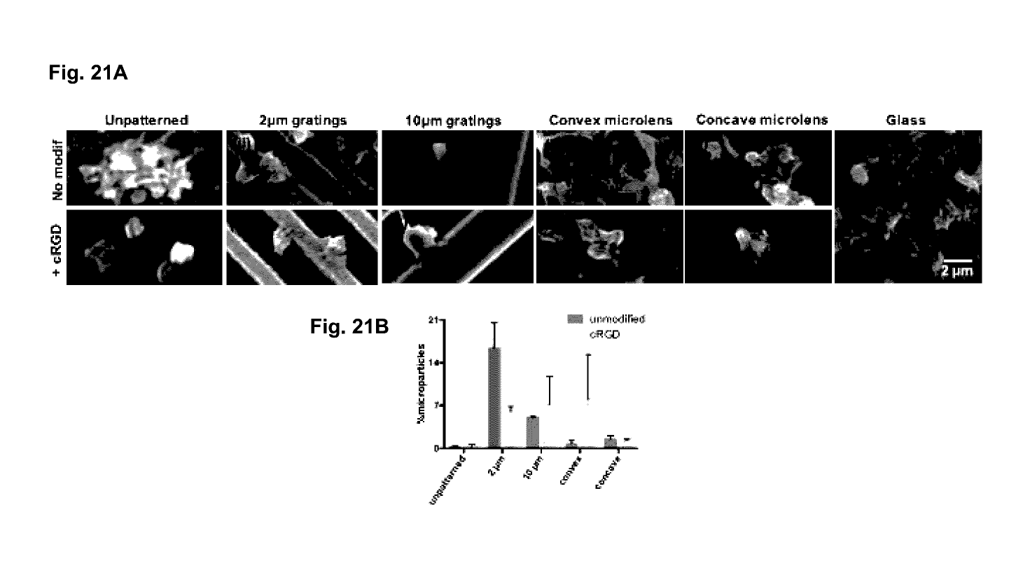

To check blood compatibility of various modified PVA, planar PVA films with various surface topographies were incubated with platelet rich plasma from rabbit. SEM showed rounded and less activated morphology of platelets attached to PVA films with topography. In contrast, platelets attached on unpatterned PVA exhibited spread morphology with fibrous extensions, similar to platelet morphology on glass (FIG. 21A(i)-(vi)). Incorporation of cRGD further improved morphology of platelets adhered onto PVA substrates (FIGS. 21A(vii)-(xi). Microparticle formation, measured by the expression of the GPIIb/IIIa marker through flow cytometry, was most notably decreased on cRGD-PVA with 2 μm gratings while it increased on cRGD-PVA with microlens (FIG. 21B). A more clinically relevant testing platform using a baboon ex vivo shunt assay also showed that under hemodynamically-relevant conditions, platelet accumulation and fibrin deposition between ePTFE and topographically- and biochemically-modified PVA grafts are statistically equivalent. Our results imply acceptable hemocompatibility of PVA and surface properties when the PVA is modified with topography or cRGD.

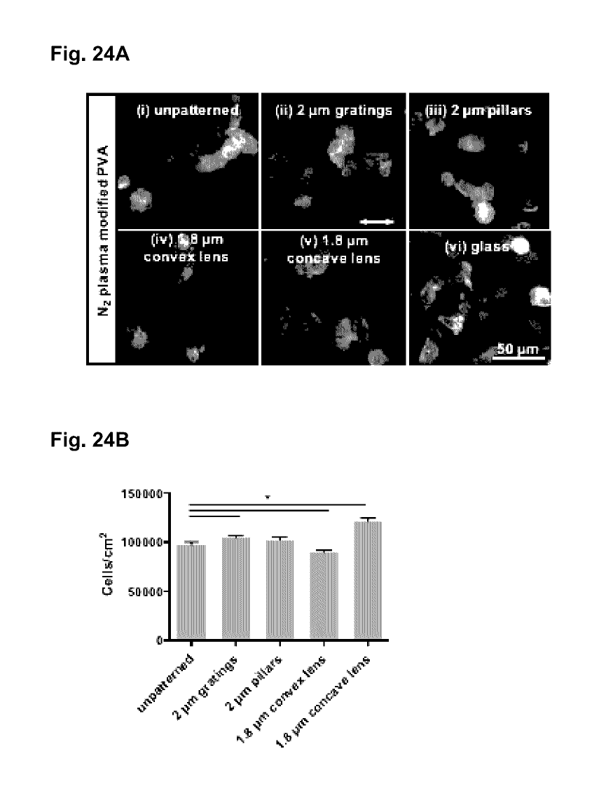

Preliminary studies on patterned cRGD-PVA films show improved attachment of endothelial cell line EAhy926 on 2 μm gratings and its modifications: 2 μm v-shaped gratings and 2 μm half-cylinder gratings (FIGS. 24A-24B).

### Fabrication of PVA-IPC Fiber Composite Tubular Scaffold

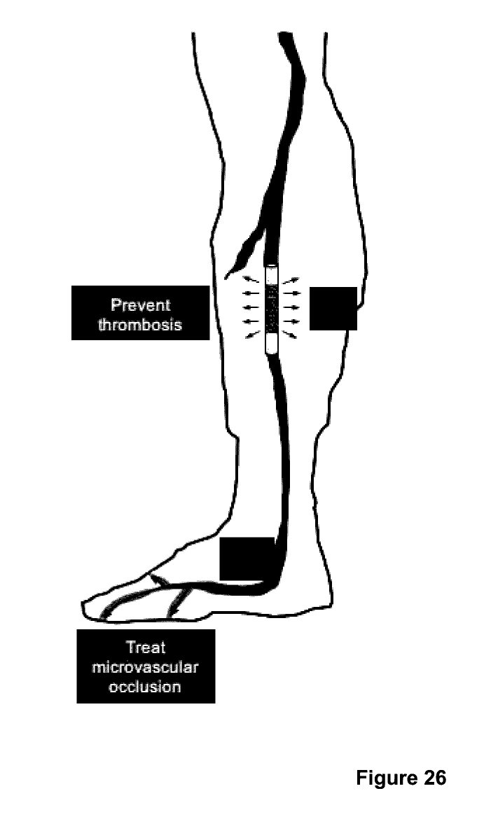

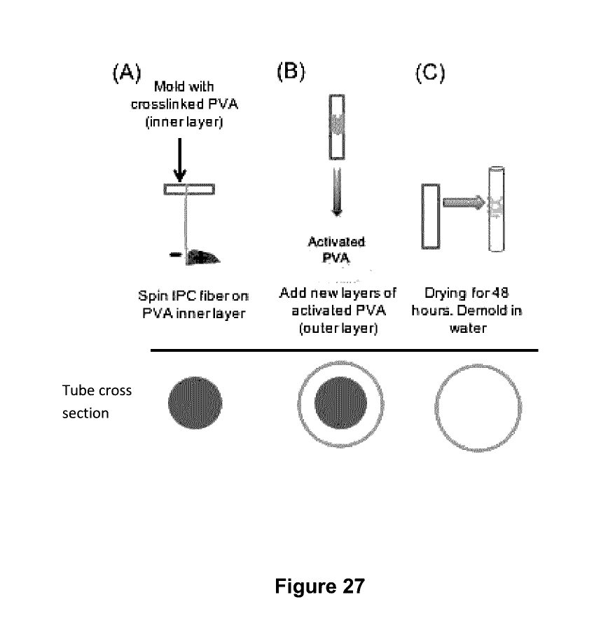

FIG. 26 highlights the advantages of a vascular graft with capability for spatially and temporally controlled release of growth factor or drugs. FIG. 27(A)-(C) shows the novel method of incorporating IPC fibers in a PVA tubular scaffold. Briefly, FIG. 27(A) shows an inner 4 layers were first cast on a tubular mold. After drying for 2 days at room temperature, IPC fibers were spun on the PVA scaffold, as will be elaborated below. A continuous strand of IPC fiber was drawn vertically from the solution interface of two oppositely charged polyelectrolytes chitosan (0.5-1% w/v, pH5.5) and alginate (0.5% to 1% w/v), with or without the addition of heparin (1%), by a set of speed-controlled rollers at 10 mm/s. After drying of the IPC fibers for 1 hour at 4° C., 2 outer layers of the PVA film were dip-casted to create an outer covering FIG. 27(B). In effect, the IPC fibers were sandwiched between two layers of PVA, thereby securing it in place.

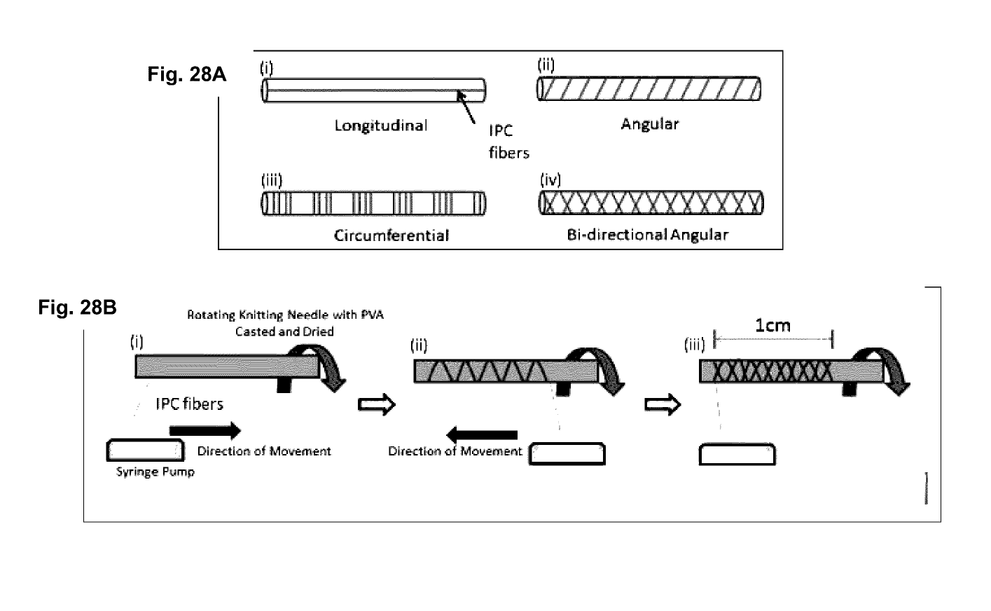

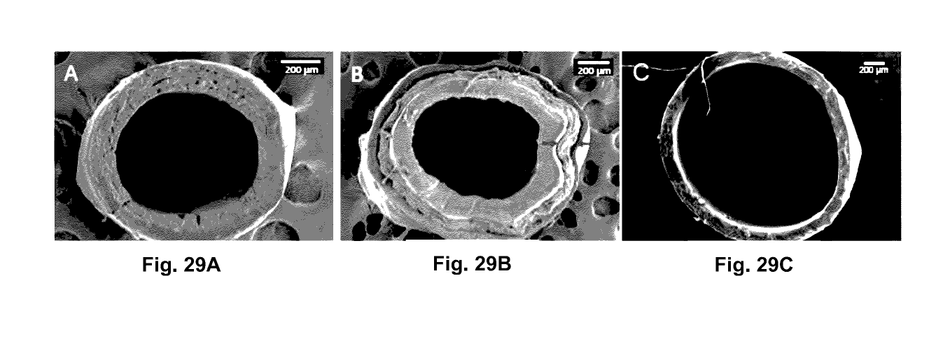

Variations in the IPC fiber spinning method may be done to orientate the fiber with respect to the PVA tubular scaffold (FIG. 28A). This serves another level of fine-tuning or tailoring the effective mechanical properties of the resulting PVA-IPC composite vascular graft. FIG. 28B shows a method for creating angular and bi-directional angular IPC fibers on to PVA scaffolds simply through the use of a programmable actuator. While rotating at a constant speed, the PVA tube was actuated laterally from the IPC fiber to generate angular IPC fibers. By actuating the PVA tube in the opposite direction, a bi-directional angular IPC fiber orientation was achieved. Controlling the rate of actuation and the rotation speed of the PVA tube were used to control the angle of the fibers with respect to the PVA tube, and thus control the mechanical property of the IPC-PVA tube. FIGS. 29A-29C show the SEM images of the PVA-IPC fiber composite tubular scaffold with different configuration of IPC fibers (FIGS. 29B, 29C) in contrast to the bare PVA tubular scaffold (FIG. 29A). Notably, despite the addition of IPC fibers into small diameter PVA scaffolds, we showed that most of the favorable mechanical properties of PVA grafts are not significantly altered by the addition of IPC fibers (Table 3).

TABLE 3
Mechanical properties of two types of PVA-IPC composite tubular scaffolds.

| | | | | | |
|---|---|---|---|---|---|
| | | Wall | Young's | | Burst |
| | Internal | thickness | modulus | Compliance | pressure |
| Scaffold type | diameter (μm) | (μm) | (kPa) | (%) | (mmHg) |

PVA-IPC composite scaffold encapsulated with VEGF

| | | | | | |
|---|---|---|---|---|---|
| Plain PVA | 1150 ± 72.93 | 312.7 ± 42.54 | 562.9 ± 144.2* | 8.171 ± 3.463 | 492.2 ± 66.88 |
| PVA-IPC | 1072 ± 73.85 | 598.4 ± 120.9 | 125.7 ± 37.78* | 4.810 ± 5.050 | 269.4 ± 134.1 |
| composite | | (part with fiber) | | | |
| Rabbit femoral | 938 ± 201 | 350-710 | 230 | 5.9 ± 0.5 | 2031-4225 |
| artery [#] | | | | | |

PVA-IPC composite scaffold encapsulated with QK-PEG

| | | | | | |
|---|---|---|---|---|---|
| Plain PVA | 2690 ± 101.2 | 375.8 ± 79.6* | 352.3 ± 32.59* | 4.88 ± 0.24 | 310.9 |
| PVA-IPC | 1963 ± 76.86 | 311.3 ± 29.0* | 830.5 ± 116.8* | 5.23 ± 2.8 | 202.6 ± 101.9 |
| composite | | | | | |
| Rabbit Aorta | 1886.5 ± 678.5 | 381.8 ± 74.8 | 10,600 ± 460 | 9.74 ± 5.44 | >2250 |

*denotes statistical significance between plain PVA scaffold and PVA-IPC composite scaffold.
# [Zheng W., et al., Biomaterials. 33, 2880-2891 (2012)].

Lysozyme Release from PVA-IPC Composite Planar Film and Composite Tubular Scaffold

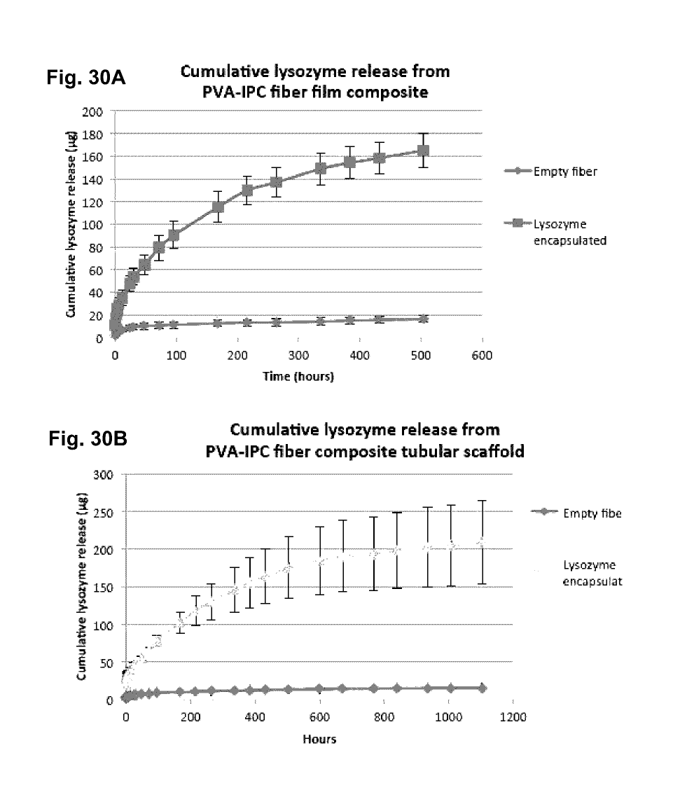

As a proof of concept, lysozyme was used as a model molecule for encapsulation into IPC fibers and incorporation into PVA planar films and PVA tubular scaffolds. Lysozyme was used as a model molecule because of its close approximation to the isoelectric point (pl) of VEGF. A total amount of 350 μg of lysozyme was incorporated through its addition to 1% chitosan and was a drawn against 1% alginate solution. After creating the composite tubular scaffold as said above, the release of lysozyme was collected using phosphate buffered saline (PBS) solution at selected timepoints. The amount of protein in each release medium collected was measured using bicinchoninic acid (BCA) assay. FIGS. 30A-30B show the sustained and controlled release of lysozyme from both types of PVA-IPC composite grafts created. This shows the potential of PVA-IPC composite scaffolds for sustained and controlled delivery of biologics.

VEGF Release from PVA-IPC Composite Tubular Scaffold

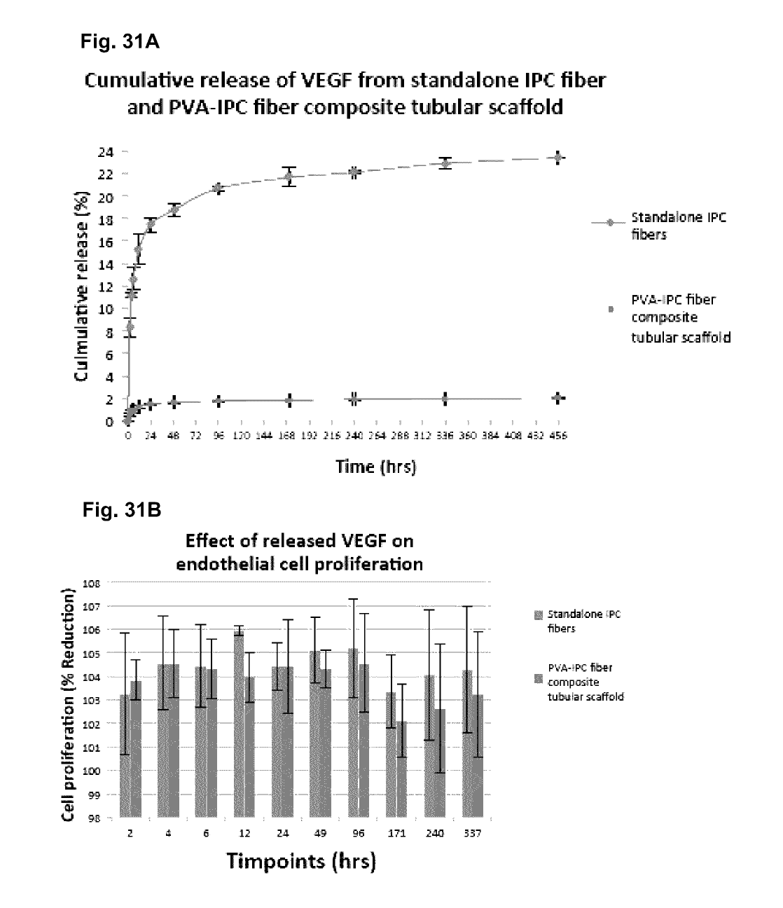

Vascular endothelial growth factor (VEGF) was also incorporated into PVA through encapsulation in IPC fibers. A total of 60 μg of the positively charged VEGF (pl˜8.3) was incorporated into the fibers by addition into the chitosan solution prior to the fiber drawing. Chitosan (1%) with VEGF was drawn against a solution of alginate (1%) with heparin (1%) (9:1 ratio). The use of heparin has been shown to decrease the initial burst release of heparin-binding growth factors, as shown previously by Liao et al [J Control Release 104(2), 347-58 (2005)]. A final concentration of 1 μg/μL was used. FIG. 31A shows the sustained release of VEGF from the PVA-IPC fiber composite tubular scaffold, in comparison to the standalone VEGF encapsulated IPC fibers. The VEGF released from the PVA-IPC composite tubular graft and the standalone IPC fibers show retained bioactivity through its ability to increase endothelial cell proliferation (FIG. 31B). Possibly, the molecular weight of VEGF (44 kDa) hindered its movement through the PVA layers after its release from the IPC fibers. In effect, a reservoir of VEGF was created in the PVA layers, thereby significantly reducing the cumulative amount of VEGF released from the IPC-PVA composite scaffold.

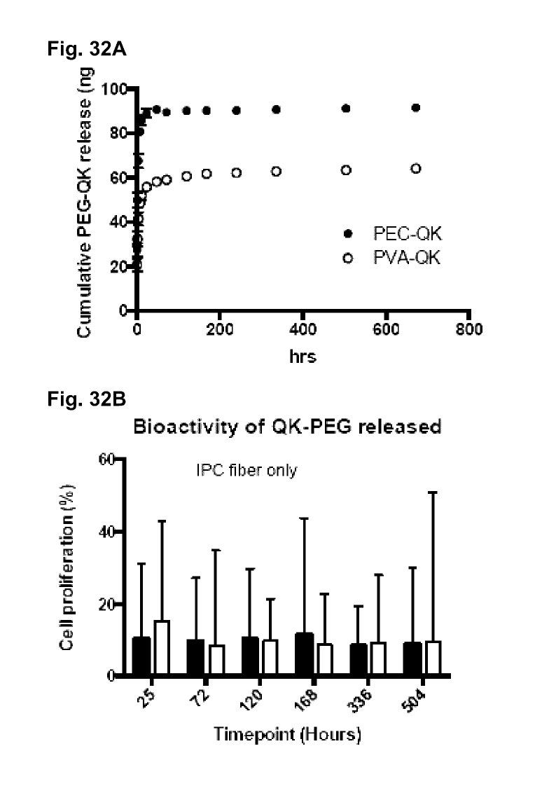

In addition to VEGF, an angiogenic peptide QK, was also encapsulated into the PVA graft. QK is a small oligopeptide that has the same mitogenic and angiogenic effects on endothelial cells as VEGF [Santulli G., et al., Journal of Translational Medicine 7(41), 1-10 (2009); D'Andrea L. D., et al., Proc. Natl. Acad. Sci. U.S.A. 102(40), 14215-20 (2005)]. QK was first PEGylated to increase its molecular weight from 1.1 kDa to 11 kDa. It was hypothesized that this molecular weight, which is close to that of lysozyme, will result in a more favorable controlled release of the growth factor. FIG. 32A shows the sustained and controlled release of QK from the PVA-IPC fiber composite tubular graft in comparison to that of the standalone IPC fibers. A higher total release was achieved for QK-PEG from IPC fibers, at a total of 64 ng in 28 days. Total release of QK-PEG from composite scaffolds was similar. For both types of scaffolds, a rapid initial release was followed by a diminished rate of release. Cumulative release profile from IPC fibers and PVA-IPC composites at each time point showed similarity, suggesting that the molecular weight and conformation of QK-PEG molecules represented the limit of diffusion through PVA hydrogel. Bioactivity of released QK-PEG was also assessed (FIG. 32B). Released QK-PEG conjugates had a positive effect on HUVEC proliferation, signifying the functionality of QK-PEG. As expected from the near-constant release rate of QK-PEG, the bioactivity of QK-PEG was also consistent at all time points examined. No significant differences were found between the HUVEC proliferation induced by release media from IPC fibers or PVA-IPC composite scaffolds.

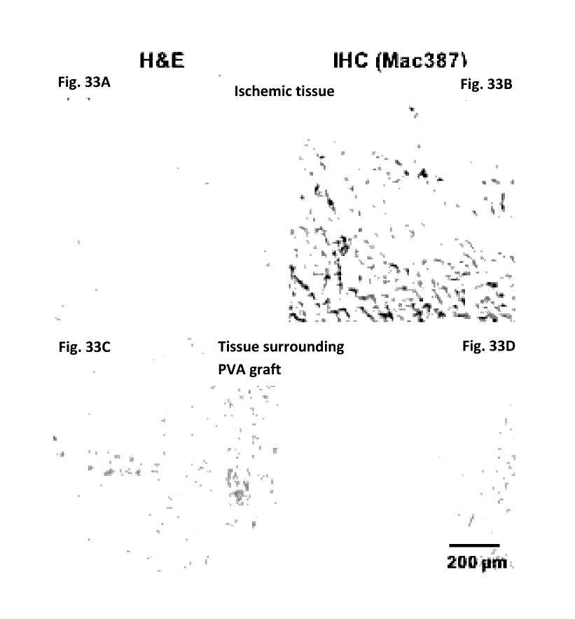

PVA-IPC grafts were implanted in the rabbit hind limb to evaluate bio inertness of the composite scaffold. Hind limb tissues were harvested 28 days after implantation and were evaluated through immunohistochemistry by detecting macrophage/monocyte infiltration (FIGS. 33A-33D). We observed that while ischemic tissue from disease rabbit model showed extensive macrophage and monocyte infiltration (FIG. 33B, brown stain against Mac387 antigen), this was not observed in tissues surrounding the PVA graft (FIG. 33D). Thus, PVA grafts did not elicit macrophage infiltration 28 days post-implantation. Our preliminary results demonstrate the biocompatibility and safety of PVA vascular graft for in vivo implantation.

The teachings of all patents, published applications and references cited herein are incorporated by reference in their entirety.

The term “comprising” is herein defined to be that where the various components, ingredients, or steps, can be conjointly employed in practicing the present invention. Accordingly, the term “comprising” encompasses the more restrictive terms “consisting essentially of” and “consisting of.”

While this invention has been particularly shown and described with references to example embodiments thereof, it will be understood by those skilled in the art that various changes in form and details may be made therein without departing from the scope of the invention encompassed by the appended claims.

### REFERENCES

Beard J. D., Which is the best revascularization for critical limb ischemia: Endovascular or open surgery?, Journal of Vascular Surgery (2008) 48, 11S-16S.
Bettinger C. J., et al., Engineering substrate topography at the micro- and nanoscale to control cell function. Angew. Chem. Int. Ed. Eng., (2009) 48, 5406.
Biela S. A., et al. Different sensitivity of human endothelial cells, smooth muscle cells and fibroblasts to topography in the nano-micro range. Acta Biomater. (2009) 5, 2460-6.
Bourke S L, et al., A photo-crosslinked poly(vinyl alcohol) hydrogel growth factor release vehicle for wound healing applications. AAPS PharmSci. (2003) 5(4), 101-11.
Brevetti G, et al., Endothelial dysfunction: A key to the pathophysiology and natural history of peripheral arterial disease? Atherosclerosis (2008) 197, 1-11.
Chaouat M, et al., A Novel Cross-linked Poly(vinyl alcohol) (PVA) for Vascular Grafts, Advanced Functional Materials (2008) 18, 2855-2861.
Curi M et al., Conduit Choice for Above-knee Femoropopliteal Bypass Grafting in Patients with Limb-threatening Ischemia, Annals of Vascular Surgery (2002) 16, 95-101.
D'Andrea L. D., et al., Targeting angiogenesis: structural characterization and biological properties of a de novo engineered VEGF mimicking peptide. Proc. Natl. Acad. Sci. U.S.A. (2005) 102(40), 14215-20.
Di Rienzo C, et al., Unveiling LOX-1 receptor interplay with nanotopography, Scientific Reports, (2013) 3, 1141-1149.
Ferrara, N., The role of VEGF in the regulation of physiological and pathological angiogenesis. EXS, (2005) 94, 209-31.
Holloway J L, et al., Analysis of the in vitro swelling behavior of poly(vinyl alcohol) hydrogels in osmotic pressure solution for soft tissue replacement. Acta Biomaterialia (2011) 7(6): 2477-82.
Hosaka, A., et al., Gelatin hydrogel microspheres enable pinpoint delivery of basic fibroblastgrowth factor for the development of functional collateral vessels. Circulation, (2004) 110(21), 3322-8.
Lamm P., et al., Autologous endothelialized vein allograft: a solution in the search for small-caliber grafts in coronary artery bypass graft operations. Circulation (2001) 18, 1108-14.
Liao, I. C., et al., Controlled release from fibers of polyelectrolyte complexes. J Control Release, (2005) 104(2), 347-58.
Liliensiek S, et al., Characterization of endothelial basement membrane nanotopography in rhesus macaque as a guide for vessel tissue engineering, Tissue Eng Part A. (2009) 15, 2643-2651.
Michaels J, Choice of material for above-knee femoropopliteal bypass graft, British Journal of Surgery (1989) 76, 7-14.
Morgan J, et al., Integration of basal topographic cues and apical shear stress in vascular endothelial cells, Biomaterials (2012) 33, 4126-4135.
Peppas N A, Merrill E W. Development of semicrystalline poly(vinyl alcohol) hydrogels for biomedical applications. J. Biomed. Mater. Res. (1977) 11(3), 423-34.
Ranjan A, Webster T J. Increased endothelial cell adhesion and elongation on micron-patterned nano-rough poly(dimethylsiloxane) films. Nanotechnology (2009) 20, 305102.
Salacinski H. J., et al. The mechanical behavior of vascular grafts: a review. Journal of Biomaterial Applications (2001) 15, 241-78.
Santulli G., et al., In vivo properties of the proangiogenic peptide QK. Journal of Translational Medicine. (2009) 7(41), 1-10.
Sarkar S., et al., Addressing thrombogenicity in vascular graft construction. Journal of Biomedical Materials Research B. Applied Biomaterials (2007) 82, 100-8.
Stewart S F, Lyman D J, Predicting the compliance of small diameter vascular grafts from uniaxial tensile tests. Journal of Biomechanics (1990) 23, 629-37.
Sun, Q., et al., Sustained vascular endothelial growth factor delivery enhances angiogenesis and perfusion in ischemic hind limb. Pharm Res (2005) 22(7), 1110-6.
Uttayarat, P., et al., Effect of Nano-to Micro-Scale Surface Topography on the Orientation of Endothelial Cells, MRS Proceedings Volume 845, 2004 Fall Meeting Symposium AA, Nanoscale Materials Science in Biology and Medicine.
Uttayarat P, et al. Microtopography and flow modulate the direction of endothelial cell migration. Am J Physiol Heart Circ Physiol. (2008) 294, H1027-35.
Venkatraman S, et al., Implanted cardiovascular polymers: Natural, synthetic and bio-inspired, Progress in Polymer Science (2008) 33, 853-874.
Yim, E. K., et al., Tissue compatibility of interfacial polyelectrolyte complexation fibrous scaffold: evaluation of blood compatibility and biocompatibility. Tissue Eng, (2007) 13(2), 423-33.
Yla-Herttuala, S., et al., Vascular endothelial growth factors: biology and current status of clinical applications in cardiovascular medicine. J Am Coll Cardiol, (2007) 49(10), 1015-26.
Zheng W., et al., Endothelialization and patency of RGD-functionalized vascular grafts in a rabbit carotid artery model. Biomaterials. (2012) 33, 2880-2891.

1. A bioactive scaffold, comprising:
poly(vinyl alcohol) scaffold and at least one modification at a surface of the scaffold, the at least one modification comprising a surface topographical cue and/or an attachment factor.

2. The bioactive scaffold of claim 1, wherein the scaffold is a small diameter vascular graft.

3. The bioactive scaffold of claim 1, wherein the surface topographical cue is an anisotropic pattern or an isotropic pattern.

4. The bioactive scaffold of claim 1, wherein the surface topographical cue is an indentation in the surface or a protrusion from the surface of the scaffold.

5. The bioactive scaffold of claim 1, wherein the surface topographical cue has a depth differential of one nanometer to 50 micrometer s relative to the surface of the scaffold.

6. The bioactive scaffold of claim 1, wherein the attachment factor is selected from the group comprising a peptide, a polysaccharide, a protein, a nucleic acid, an oligonucleotide, a nanoparticle, an organic small molecule, or an inorganic compound.

7. The bioactive scaffold of claim 6, wherein the attachment factor is selected from the group comprising heparin, fibronectin, Arg-Gly-Asp-Ser (RGDS) or cyclo(Cys-Arg-Arg-Gly-Asp-Trp-Leu-Cys) (cRGD).

8. The bioactive scaffold of claim 1, further comprising an interfacial polyelectrolyte complexation (IPC) fiber and a biologic.

9. A bioactive scaffold comprising poly(vinyl alcohol), a IPC fiber and a biologic.

10. The bioactive scaffold of claim 9, wherein the biologic is encapsulated in a matrix comprising the IPC fiber, such that, in use, the matrix sustainably and controllably releases the biologic.

11. The bioactive scaffold of claim 9, wherein a matrix comprising the IPC fiber is enclosed between one or more layers of poly(vinyl alcohol).

12. The bioactive scaffold of claim 9, wherein the biologic is selected from the group comprising a growth factor, an enzyme, a peptide, a cell, an antibody, an antioxidant, an angiogenic molecule, an antiangiogenic molecule, an immune-modulatory molecule, a pro-inflammatory molecule, an anti-inflammatory molecule, a nucleic acid, an oligonucleotide, an adhesion molecule, or a pharmaceutical composition.

13. The bioactive scaffold of claim 12, wherein the biologic is vascular endothelial growth factor (VEGF).

14. A method for controlling the release of a biologic from a bioactive scaffold, comprising:
providing a bioactive scaffold comprising poly(vinyl alcohol), a IPC fiber and a biologic, wherein the composition and fabrication of the scaffold is selected to control the release of the biologic; and
exposing the bioactive scaffold to conditions in which release of the biologic is induced.

15. A method of fabricating a bioactive scaffold, comprising:
(a) providing a tubular mold, optionally comprising a topographically patterned outer surface;
(b) contacting the tubular mold of step (a) with an aqueous solution comprising poly(vinyl alcohol) to form a tube coated with poly(vinyl alcohol);
(c) drying the coated tube of step (b) to evaporate residual water;
(d) repeating steps (b) and (c) as necessary to achieve a desired thickness of the bioactive scaffold.

16. The method of claim 15, additionally comprising steps (e) and (f), wherein steps (e) and (f) are performed after any iteration of step (c), and further wherein steps (e) and (f) are:
(e) wrapping a fibrous matrix comprising IPC fibers onto the outer surface of the coated tube to form a fiber-wrapped coated tube; and
(f) contacting the fiber-wrapped coated tube of step (e) with an aqueous solution comprising poly(vinyl alcohol) to form a layered tube with an outer coating of poly(vinyl alcohol).

17. The method of claim 16, wherein the fibrous matrix further comprises a biologic.

18. The method of claim 17, wherein the biologic is vascular endothelial growth factor (VEGF).

19. A bioactive scaffold produced according to the method of claim 15.

20. A method of treatment comprising administering to a subject in need of such treatment a bioactive scaffold as defined in claim 1.

21. The method of claim 20, wherein the treatment is vascular repair.

22. A kit for vascular repair comprising a bioactive scaffold as defined in claim 1.
